(19) United States
(12) Patent Application Publication
(10) Pub. No.: US 2012/0116285 A1
(43) Pub. Date: May 10, 2012

(54) DEVICES FOR TREATING OBESITY AND METHODS OF USING THOSE DEVICES

(76) Inventor: Chandra S. Duggirala, Walnut Creek, CA (US)

(21) Appl. No.: 13/133,040

(22) PCT Filed: Dec. 7, 2009
(86) PCT No.: PCT/US2009/006440

§ 371 Date: Jan. 23, 2012

Related U.S. Application Data
(60) Provisional application No. 61/140,933, filed on Dec. 27, 2008.
(60) Provisional application No. 61/165,313, filed on Mar. 31, 2009.
(60) Provisional application No. 61/279,967, filed on Oct. 27, 2009.

(51) Int. Cl. A61F 2/04

(52) U.S. Cl. 604/8

(57) ABSTRACT

Described here are devices for treating obesity. The devices are situated in the stomach and duodenum and maintain separation of the chyme stream leaving the stomach from the stream containing bile and pancreatic fluids exiting the Ampulla of Vater until well down into the small intestine. The devices, however, permit other digestive fluids to enter the chyme stream and hormones to enter the blood stream.

### FIELD

Described here are devices for treating obesity. The devices are situated in the stomach and duodenum and maintain separation of the chyme stream leaving the stomach from the stream containing bile and pancreatic fluids exiting the Ampulla of Vater until well down into the small intestine. The devices, however, permit other digestive fluids to enter the chyme stream and hormones to enter the blood stream

### BACKGROUND

Obesity continues to increase in importance as a major health problem. In addition to the obvious strains on the back, the musculature, and other structures of the human body, obesity affects the body's organs, particularly the heart and circulatory systems, via hypertension and coronary artery disease. Obesity contributes to an estimated half-million deaths a year along with co-morbidities like Type-II diabetes.

Obesity is a complex disorder. Nevertheless, the medical consensus is that the cause is simply a combination of an increase in the intake of excessive calories and a reduction in energy expenditure. Although the treatments seem intuitive, they are not easily instituted nor maintained. Dieting is not an effective long-term solution for most obesity disorders. Once an individual has slipped past the BMI of 30, more drastic solutions are often required.

There are several invasive procedures for reducing consumption and producing long-term weight loss. Two common surgical procedures are the Roux-en-Y gastric bypass and the biliopancreatic diversion with duodenal switch (BPD). Both procedures reduce the size of the stomach and shorten the effective length of intestine available for nutrient absorption. Reduction of the stomach size reduces stomach capacity and the ability of the patient to take in food.

In the BPD procedure, large lengths of jejunum are bypassed resulting in malabsorption and therefore, reduced caloric uptake. In the BPD procedure, the stomach is not reduced in size as much in the Roux-en-Y gastric bypass procedure so that the patient is able to consume sufficient quantities of food to compensate for the reduced absorption. The latter procedure is reserved for the most morbidly obese as there are several serious side effects of prolonged malabsorption.

Interestingly, these procedures also have immediate but therapeutic effect on diabetes II.

These surgical procedures have some detrimental effects: bypassing the duodenum causes difficulty in digesting fatty, sugary, and complex carbohydrate-rich foods and, should a person eat those foods, that digestion causes a “dumping” syndrome. Dumping occurs when carbohydrates directly enter the jejunum without being first conditioned in the duodenum. That bypassing causes the intestinal lining to discharge a large quantity of fluid into that food. The total effect on the patient is light-headedness and a severe diarrhea.

Although the cause-and-effect seems straightforward, their exact mechanism of is not well understood. Eventually patients learn that compliance with the dietary restrictions imposed by their modified anatomy alleviates the light-headedness and dumping.

The morbidity rate for these surgical procedures is comparatively with 11% requiring surgical intervention for correction. Early small bowel obstruction occurs at a rate of between 2 to 6% in these surgeries and mortality rates are reported to be approximately 0.5 to 4%. Although surgery seems to be an effective answer, the complication rates associated with current invasive procedures are quite high.

Laparoscopic techniques applied to these surgeries provide faster recovery but still carry significant risks, particularly for very ill patients, and require high skill levels the surgeon.

Devices to reduce absorption in the small intestines have been proposed (See U.S. Pat. No. 5,820,584 (Crabb), U.S. Pat. No. 5,306,300 (Berry) and U.S. Pat. No. 4,315,509 (Smit)).

### BRIEF DESCRIPTION OF THE DRAWINGS

FIG. 1 provides a partial cutaway view of the digestive tract between the esophagus and the small intestine.

FIG. 2 provides a cutaway view of the Ampulla of Vater in the duodenum.

FIG. 3 provides a partial perspective view of the digestive tract between the esophagus and the anus.

FIG. 4 provides a schematic perspective view of my device showing its component sections.

FIG. 5 provides a schematic, perspective, partial cutaway view of my device and its typical placement in the duodenum.

FIGS. 6A to 6F show examples of physical affixing components or adhesives that are especially suitable for fixing the upper section to the pylorus or to the stomach wall.

FIG. 7 shows one variation of the upper section where the section is a continuous membrane conforming in general shape to the pylorus.

FIG. 8A shows a partial sectional view of another variation of an upper section having stiffeners to maintain the shape of the continuous membrane against the surrounding pylorus.

FIG. 8B is a partial sectional view of the upper section variation shown in FIG. 8A showing, in particular, the stiffeners in position in the continuous membrane.

FIGS. 9A-9B show a variation of the upper section in comprising a bare expandable stent-like structure that may be affixed in the pylorus or proximal of the pylorus in the stomach.

FIG. 10A shows implantation of the upper section variation shown in FIGS. 9A and 9B into the proximal pylorus.

FIG. 10B shows placement of the stent-like structure into the pylorus such that the open framework extends past the pylorus and leaves open framework structure in the duodenum.

FIG. 10C shows optional securement of the stent-like structure to the pylorus with a fastener such as a suture.

FIGS. 11A-11C show another variation of an upper structure having a stent-like structure with an open wire framework, optional continuous membrane, and barbs that act as fasteners to the muscle of the pylorus.

FIGS. 12A and 12B show additional variations of the upper section utilizing stent-like structures.

FIGS. 13 and 14 show variations of the upper section having sealing components proximal and distal of the pylorus.

FIG. 15 shows another variation of the upper section.

FIG. 16 shows another variation of the upper section, but in this instance having a donut-shaped inflatable or inflated component that is operable to occupy a volume in the stomach and further to assist in treating obesity.

FIGS. 17A and 17B show, respectively, a perspective view and a side view of another variation of an upper section having ancillary volume-filling inflatable components attached to the membrane.

FIG. 18 shows a side, cross-section view of my device having an upper section, a separator section, a lower seal section, and a conduit section.

FIGS. 19 and 20 show variations of the upper section utilizing magnetic rings to fix the device in place.

FIGS. 21A and 21B show, respectively, side cross-section and perspective views of another variation of an upper section having a pair of biased valve leaves that stay closed until a design pressure is found upon the valve leaves.

FIGS. 22A and 22B show, respectively, side view cross-sectional views of another variation of an upper section having a biased valve that stays closed until a design pressure is found upon the valve.

FIGS. 23A and 23B show, respectively, side cross-section and perspective views of another variation of an upper section having an orifice with a size selected to provide a continuing flow of chyme.

FIG. 24 shows a side cross-section of an upper section having a circular seal comprising a compressible, resilient, polymeric foam that seals the upper section wall against the pylorus.

FIG. 25 shows a side cross-sectional view of another variation of an upper section.

FIGS. 26A and 26B show, respectively, a side, cross-section view and a top, cross-section view of one variation of a separator section.

FIGS. 27A and 27B show, respectively, a side, cross-section view and a top, cross-section view of one variation of a separator section and its relationship to a lower sealing section.

FIGS. 28A and 28B show, respectively, a side, cross-section view and a top, cross-section view of another variation of a separator section and its relationship to a lower sealing section.

FIGS. 29A and 29B show, respectively, a side, cross-section view and a top, cross-section view of another variation of a separator section.

FIGS. 30A and 30B show, respectively, a side, cross-section view and a top, cross-section view of another variation of a separator section.

FIGS. 31A and 31B show, respectively, a side, cross-section view and a top, cross-section view of another variation of a separator section.

FIGS. 32A, 32B, 33, and 34 show variations of the separator section.

FIGS. 35A, 35B, and 35C show, respectively, a side, cross-section view, a top, cross-section view, and a side view of another separator section variation.

FIGS. 36A and 36B show respectively, a side, cross-section view and a top, cross-section view of another variation of a separator section and its relationship to a conduit section comprising multiple conduits.

FIGS. 37A and 37B show, respectively, a side view and a cross section top view of another variation of a separator section and its relationship to a conduit section.

FIGS. 38A and 38B show, respectively, a side, cross-section view and a top, cross-section view of another variation of a separator section.

FIGS. 39A and 39B show, respectively, a side, cross-section view and a top, cross-section view of another variation of a separator section having a separator wall, the interior of which defines a chyme passageway.

FIGS. 40A to 43B show various ways of affixing blister-shaped separator section walls to the duodenal walls.

FIG. 44 shows a side, cross-section of another variation of a separator section.

FIG. 45 is a side view, cross-section of a separator section having bellows in the separator wall allowing the accumulator volume to expand as the bile and pancreatic fluids exiting the Ampulla of Vater pass into that volume.

FIG. 46 shows a separator section before and after expansion in a duodenum.

FIGS. 47A and 47B show, respectively, an exploded perspective view of a separator section and a top view of the assembled separator section.

FIG. 48-50 show variations of the separator section.

FIGS. 51-63 show various seal configurations for use in the lower seal section.

FIG. 64-66 show variations of lower seal structures.

FIGS. 67 and 68 show quick disconnect snap conduit connection assemblies.

FIG. 69 shows a connection assembly having a magnetic base portion with a mating surface with multiple connector barbs for connecting the base portion to the duodenal wall.

FIG. 70 shows another variation of a magnetic connection assembly having a base portion and a removable portion connected to the conduit member.

FIGS. 71A and 71B show a base support.

FIGS. 72 to 79 show variations of the conduit that may be fixed to the other portions of the device or duodenal wall as otherwise discussed here or may be detachable.

FIGS. 79 and 80A-80C show variations of conduit members.

FIGS. 81A-81D show guide member based devices for introducing conduit members into the small intestine.

FIGS. 82A-82G schematically depict a method for implanting my device.

FIGS. 83A1, 83A2, and 83B show an integral balloon based component for transporting a conduit member to the small intestine.

FIGS. 84A, 84B, 84C1, and 84C2 show devices for delivering a conduit member to the small intestine.

FIGS. 85A-85C show various connectors for temporarily attaching a conduit to an endoscope.

### DETAILED DESCRIPTION

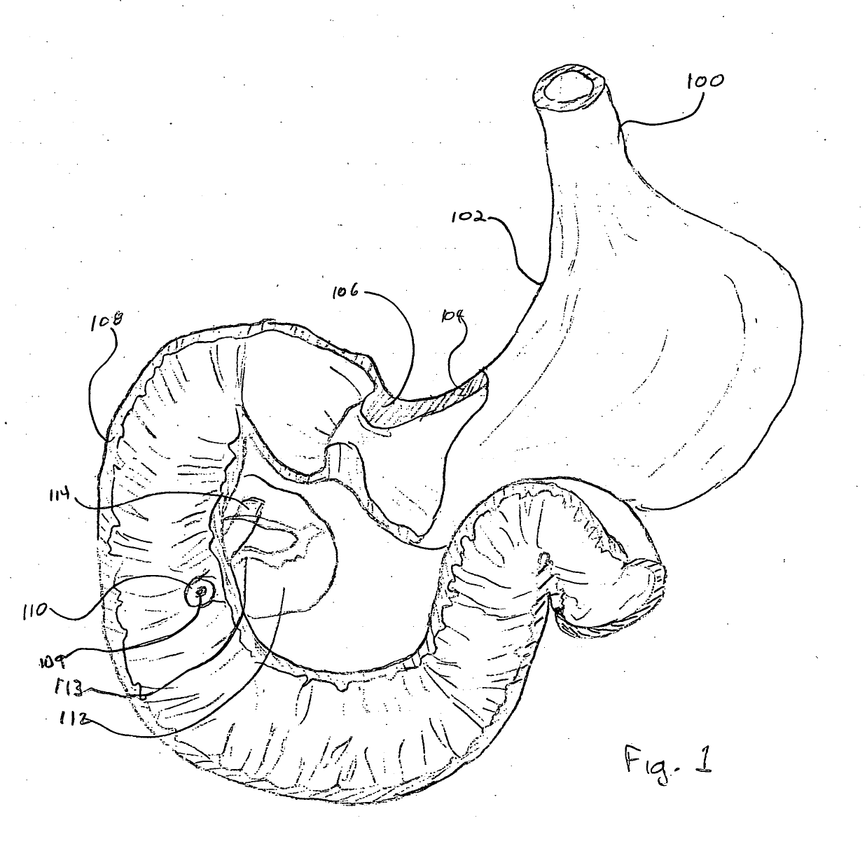

FIG. 1 shows a cutaway view of a portion of the digestive tract. Digestion begins in the mouth. Chewing cuts and grinds ingested food into pieces for passage through the throat or pharynx and esophagus. Saliva mixing with that food provides both a transporting fluid for such passage and begins the chemical breakdown of the food.

The esophagus extends to the stomach and transports food to that organ by a coordinated series of muscular contractions called peristalsis. The lower esophageal sphincter (100) is located at the junction of the esophagus and the upper end of the stomach (102) and provides a region of comparatively high pressure and functions as a one-way valve resisting food back-flow from the stomach into the esophagus while allowing or causing food movement into the stomach.

The stomach (102) is a sac-like organ with strong muscular walls (104) having a relatively complex operation. In addition to holding food, the stomach also mixes and grinds it. The stomach secretes acids and enzymes that continue to chemically and physically break down the food. The stomach operates in a semi-batch mode—small uneven masses of food enter the stomach and are held there and manipulated there by peristalsis until the size of those food particles normalizes and attains a size of about one to two millimeters. After the food particles in the stomach (102) reach that size, the pylorus (106) opens and the food slurry containing those particles—the slurry is an acidic mixture called chyme—passes into the first section of the small intestine, the duodenum (108).

The duodenum (108) continues the breakdown of the food particles by mixing the chyme with enzymatic materials issuing through the muscular valve known as the Sphincter of Oddi (109) through the Ampulla of Vater (110) into the second part of the duodenum (108). The Sphincter of Oddi (109) is relaxed by the hormone cholecystokinin (CCK) via vasoactive intestinal polypeptide (VIP). The Ampulla of Vater (110) typically excretes enzymes from the pancreas (112) and, via the common bile duct (114), bile from the gallbladder. In some individuals, the pancreatic duct and the common bile duct (114) are not joined and have separate openings into the duodenum (108).

Bile aids in the digestion of fats and neutralizes acid from the stomach (102). Pancreatic enzymes break down proteins, fats, and carbohydrates.

Bile is produced in the liver. The liver and pancreas further add an alkaline watery solution rich in bicarbonates that both dilutes the bile solution and increases its alkalinity. Bile flows either to the duodenum or to the gallbladder into the common hepatic duct, which joins with the cystic duct from the gallbladder to form the common bile duct (104). The common bile duct in turn joins with the pancreatic duct to empty into the duodenum. If the Sphincter of Oddi (109) is closed, bile flows into the gallbladder, where it is stored and concentrated. This concentration occurs via the removal of or absorption of water and small electrolytes. The bile retains the original organic molecules. Cholesterol is also released with the bile, dissolved in the acids and fats found in the concentrated solution. When chyme is released by the stomach (102) into the duodenum (108), the duodenum releases cholecystokinin, which in turn causes the gallbladder to release the concentrated bile.

The liver can produce up to one liter of bile per day. Most of the salts secreted in bile are reabsorbed in the terminal Ileum and re-used. Blood from the Ileum flows directly to the hepatic portal vein and returns to the liver for reabsorption and re-use.

Bile has surfactant activity, helping to emulsify fats for improved absorption in the small intestine. Bile salts, i.e., salts of taurocholic acid and deoxycholic acid, combine with phospholipids to break down fat globules during that emulsification. The resulting emulsified droplets are micellar providing increased surface area and absorption. Pancreatic lipase acts upon the fat triglycerides in the small intestine and breaks them down into fatty acids and monoglycerides. These products are absorbed by the intestinal villus.

Since bile increases the absorption of fats, it is an important part of the absorption of the fat-soluble vitamins such as vitamins A, D, E, and K.

In addition to its function during digestion, bile carries hemoglobin breakdown products, e.g., bilirubin, produced in the liver and neutralizes the stomach acid before it enters the Ileum, the final section of the small intestine. Bile salts also have a bacteriocidal function and act upon certain bacteria entering with the food.

Pancreatic fluids passing through the Sphincter of Oddi (109) in the Ampulla of Vater (110) and into the second part of the duodenum (108) are a soup of digestive enzymes, bicarbonates, and salts. Digestive enzymes include trypsin (a protease that cleaves proteins into basic amino acids), chymotrypsin (a protease that cleaves proteins into aromatic amino acids), carboxypeptidase (a protease that cleaves the terminal acid group from a protein), pancreatic lipase, steapsin (degrades triglycerides into fatty acids and glycerol), and pancreatic amylase that, in addition to degrading starch, glycogen, and cellulose, also degrades most other carbohydrates.

Not incidentally, the pancreas (112) is also a gland organ and a component of the endocrine system. It produces several important hormones, including insulin, glucagon, and somatostatin, and passes those hormones into the blood.

FIG. 2 shows a close-up cross-section of the duodenum (108) in the vicinity of the Ampulla of Vater (110). The central location of the muscular Sphincter of Oddi (109) passing through the Ampulla of Vater (110) into the duodenum (108) may be seen. The Sphincter of Oddi (109) is relaxed by the hormone cholecystokinin (CCK) via vasoactive intestinal polypeptide (VIP). The Sphincter of Oddi (109) is seen to be connected to the pancreatic duct (113) and, via the common bile duct (114), the gallbladder.

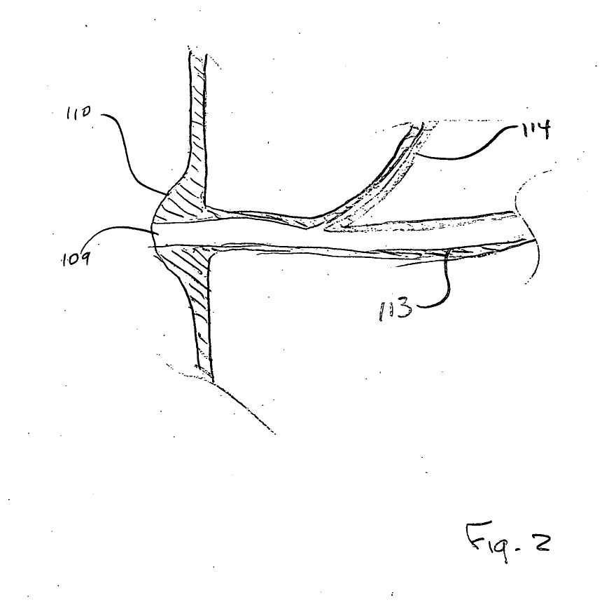

Referring to FIG. 3, as mentioned just above, the small intestine (120) is made up of a long section of tubing loosely coiled in the abdomen and having three segments—the duodenum (108), jejunum (122), and the (124) Ileum. Peristalsis moves chyme through the small intestine (120) and mixes it with digestive secretions. The duodenum (108) is largely responsible for continuing the process of breaking down food, with the jejunum (122) and the (124) Ileum being mainly responsible for the absorption of nutrients into the bloodstream.

Once the nutrients have been absorbed and the leftover liquid has passed through the small intestine (120), the remainder is passed to the large intestine, or colon (130). The colon (130) is a long muscular organ that connects the small intestine (130) to the rectum (140). It is made up of the ascending colon (132), the transverse colon (134), the descending colon (136), and the sigmoid colon (138) that connects to the rectum. Waste remaining after completion of the digestive process, passes through the colon (130) by means of peristalsis, first in a liquid state and ultimately in solid form. As it passes through the colon (130), the colon (130) removes most of the remaining water. The waste, mostly food debris and bacteria, is stored in the sigmoid colon (138) until it passes into the rectum (140). When the descending colon (136) becomes full of stool, or feces, it empties its contents into the rectum (140) to begin the process of elimination.

The rectum (140) is a short chamber that connects the colon (130) to the anus (144). It receives waste from the colon (130) and holds it until evacuation. Typically, neurosensors detect the presence of feces in the rectum (140). The rectum (140) is voided through the anus (144) when the anal sphincters (146) relax and the rectum (140) contracts.

The anus (144) is the distal-most portion of the digestive tract. It is made up of the pelvic floor muscles and the two anal sphincters (internal and external muscles) (146). The pelvic floor muscle creates an angle between the rectum (140) and the anus (144) to maintain waste in the rectum (140). The internal sphincter (146) is always tight, except when feces enters the rectum (140).

FIGS. 4 and 5 show the four major portions of my device (200) and their general relationship to the digestive tract.

As shown in FIG. 4, my device (200) comprises a central body (201) in turn comprising four sections: a.) an upper section (202) typically supporting the device (200) and substantially sealing the device (200) against the wall of the digestive tract, e.g., within the stomach, pylorus, or duodenum, b.) a separator section (206) that substantially maintains separation between the chyme inside the device (200) and fluids such as bile and pancreatic fluids situated outside the device, c.) one or more lower sealing sections (208) operable to maintain at least one volume defined additionally by the digestive tract wall, the upper section (202), and the separator section (206), the volume operative for collecting (and optionally storing) bile and pancreatic fluids expressed from the Ampulla of Vater (110), and maintaining a separation between the chyme inside the device (200) and fluids such as bile and pancreatic fluids situated outside the device (200), and d.) a conduit section (210) comprising one or more conduits (212) in fluid communication with the collection volume exterior to the device (200) for transporting the separated bile and pancreatic fluids to or towards the Ileum (124) for release there.

The functions of certain of the sections may be made redundant in the device, e.g., sealing functions may be placed in the upper section (202) and in the lower sealing section (208) or may be additionally placed in the separator section (206) to complement the sealing functions in those sections. The functions of certain of the sections may be transferred to other sections as described in detail below.

The structure of certain variations of the device (200) may render unnecessary a separate component to attain a specifically listed function. For instance, the structure of a component used to affix the device (200) to a digestive tract wall may also function to seal the device to that wall rendering a separate sealing component redundant or unnecessary.

FIG. 5 shows the typical placement of my device (200) in the digestive tract. The device (200) extends from the stomach (102) in the region of the pylorus (106), through the pylorus (106), through at least a portion of the duodenum (108)—specifically past the Ampulla of Vater (110)—and into the Ileum section (124) of the small intestine (130).

In the variation of my device schematically depicted in FIG. 5, the upper section (202) of the device (200) resides in the stomach (102) and may be fixed to the pylorus (106). The device (200) may be sealed to the wall of the stomach (102) or against the pylorus (106) or against the wall of the duodenum (108). By “sealing” is meant that substantially no chyme, in particular, less 2-3% of the chyme passing out of the stomach (102) over a particular elapsed time period, passes exterior to the device (200) by the region having the sealing function. Alternatively, by “sealing” is meant that substantially no bile or pancreatic enzymes, in particular, less 2-3% of the bile or pancreatic enzymes passing out of the Ampulla of Vater (110) over a particular elapsed time period, passes interior to the device (200) by the region having the sealing function. The pylorus (106) is an especially advantageous site for affixing the device (200) to the digestive tract in that the pylorus (106) is a thick, muscular member that readily accepts such affixing components, serves as an excellent site for anchoring devices and maintains the position of the device (200) over extended periods of time.

As will be explained below, the upper section (202) may include other ancillary components or perform functions ancillary or auxiliary to the fixation function, e.g., provide temporary stomach volume reduction, exude drugs for treatment, and slow or delay passage of chyme through the opening or passageway (204) in the device (200), thus causing a delay in emptying of the stomach.

The separator section (206) has a pair of major functions: 1.) collecting bile and pancreatic enzymes passing out of the Ampulla of Vater (110) and 2.) maintaining substantial separation between a.) the bile and pancreatic enzymes stream passing from the Ampulla of Vater (110) from b.) the chyme interior to the separator section (206) over the area of the separator section (206). The collection and separation functions of the separator section (206) do not mandate a specific shape, length, or area save those necessary to collect bile and pancreatic enzymes and maintain substantial separation of that collective fluid stream from chyme.

The separator section (206) may include other ancillary components or perform functions ancillary or auxiliary to the collection and separation functions, e.g., provide temporary storage of the bile and pancreatic enzymes or provide slowed or delayed passage of chyme through the opening (204) in the device (200) in the region of the separator section (206) or provide a sealing function with the digestive tract wall proximal of the Ampulla of Vater (110). The temporary storage of bile and pancreatic enzymes may be for a variety of different reasons, e.g., delaying release of bile and pancreatic enzymes into a region of the Ileum (124) until a majority of chyme has passed through that region of the small intestine (130).

The region of the separator section (206) that is in contact with the separated bile and pancreatic fluids is also in hydraulic or fluid communication with the one or more conduits (210) that transports the separated bile and pancreatic fluids to or towards the Ileum (124) for release there.

The device (200) also comprises one or more lower sealing sections (208) operable to maintain at least one volume defined additionally by the digestive tract wall, the upper section (202), and the separator section (206). Said in another way, one or more lower sealing sections (208) defines a lower surface of the volume operative for collecting (and optionally storing) bile and pancreatic fluids expressed from the Ampulla of Vater (110) and maintaining a separation between the chyme inside the one or more passageways (204) of the device (200) and fluids such as bile and pancreatic fluids situated outside the device (200).

The major functions of the lower section (208) are to seal the device (200) to the digestive tract wall and to maintain substantial separation between a.) the bile and pancreatic enzymes stream passing from the Ampulla of Vater (110) from b.) the chyme passing by or through the interior (204) of the separator section (206) into the lower section (208). Although the lower section (208) may also be affixed to the digestive tract wall, such fixation is a secondary function.

As is the case with the upper section (204), by “sealing” is meant that substantially no chyme, in particular, less 2-3% of the chyme passing out of the stomach (102) over a particular elapsed time period, passes exterior to the device (200) in the region of the lower section (208) having the sealing function. Alternatively, by “sealing” is meant that substantially no bile or pancreatic enzymes, in particular, less 2-3% of the bile or pancreatic enzymes passing out of the Ampulla of Vater (110) over a particular elapsed time period, passes interior to the device (200) in the region of the lower section (208) having the sealing function.

The device (200) may comprise inner and outer continuous surfaces extending from the proximal end of the upper section (202) to the distal end of the lower section (208) or may have openings of small or substantial size in that interval.

The conduit section (210) comprises one or more tubular conduits (212) operable to transport the bile and pancreatic enzymes collected in the volume of the separator section (206) operative to collect those fluids, for a selected distance into the Ileum (124) and to emit them there. The length of the tubular conduits (212) is selected to traverse the selected distance or distances. The tubular conduits (212) need not be of the same length. The number of tubular conduits (212) may be from one to a dozen or more. The tubular conduits (212) may be of any convenient cross section, e.g., having round, oval, square, triangular, or other shaped single or multiple passageways, may have continuous, non-continuous, solid, partially porous, or otherwise configured walls. The tubular conduits (212) may have openings at selected sites, e.g., at the more distal ends of the tubular conduits (212).

### Upper Section

As noted above, my device (200) comprises four generally distinct sections, the most proximal of which is the upper section (202). The upper section (202) may be affixed to the pylorus (106) within the stomach (102) or distanced away from the pylorus (106) in the wall of the stomach (102). The pylorus (106) is a sturdy and thick muscle that provides a sturdy support for the device (200).

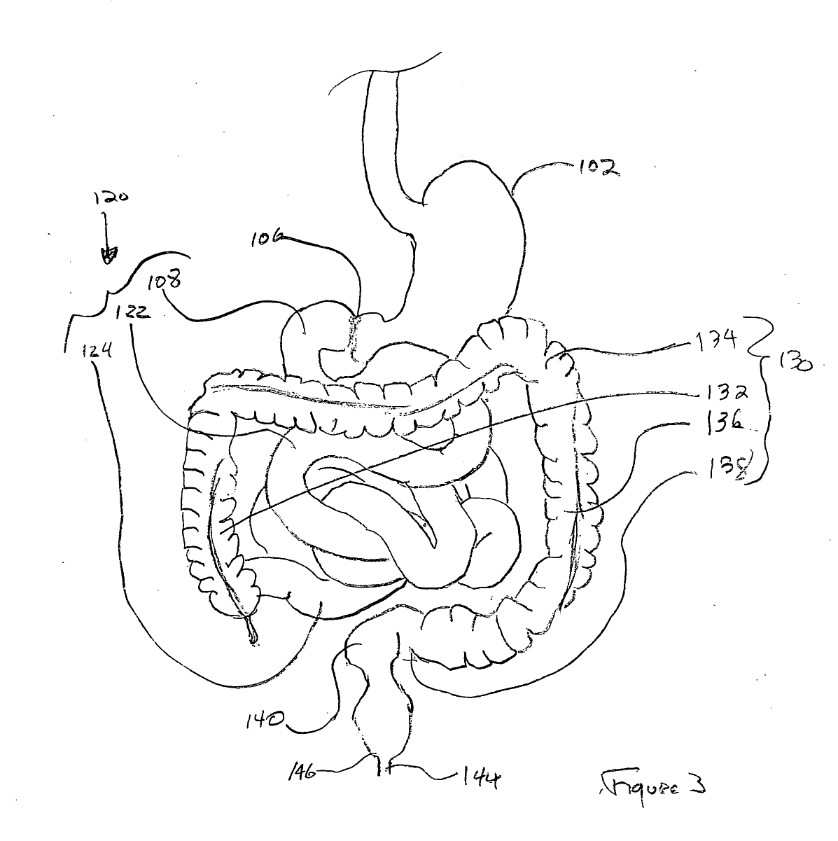

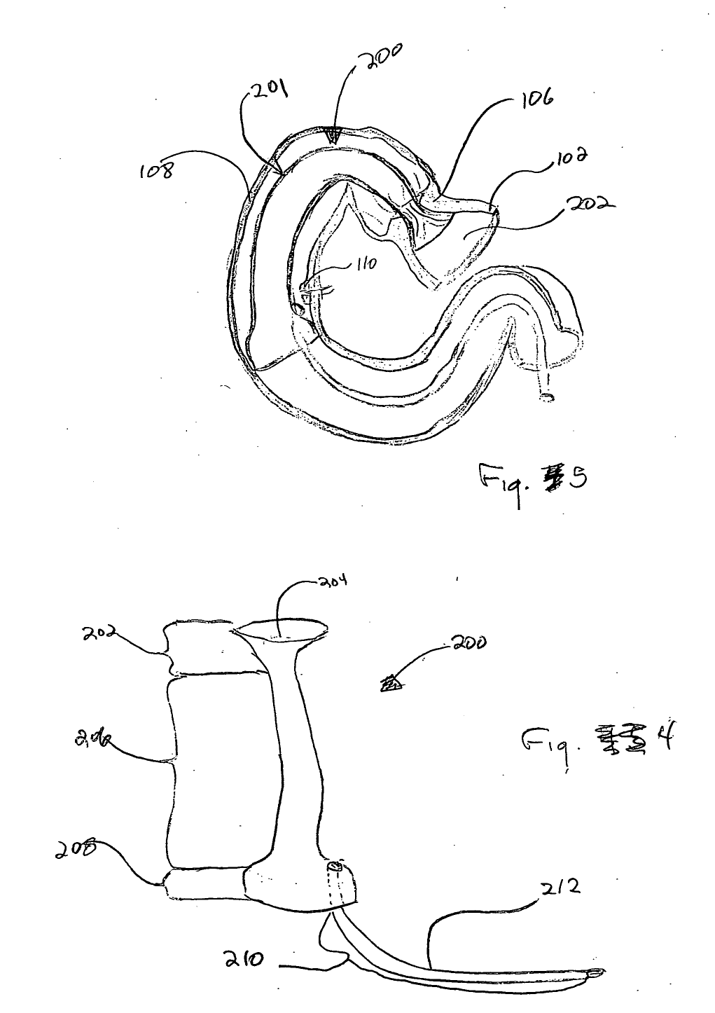

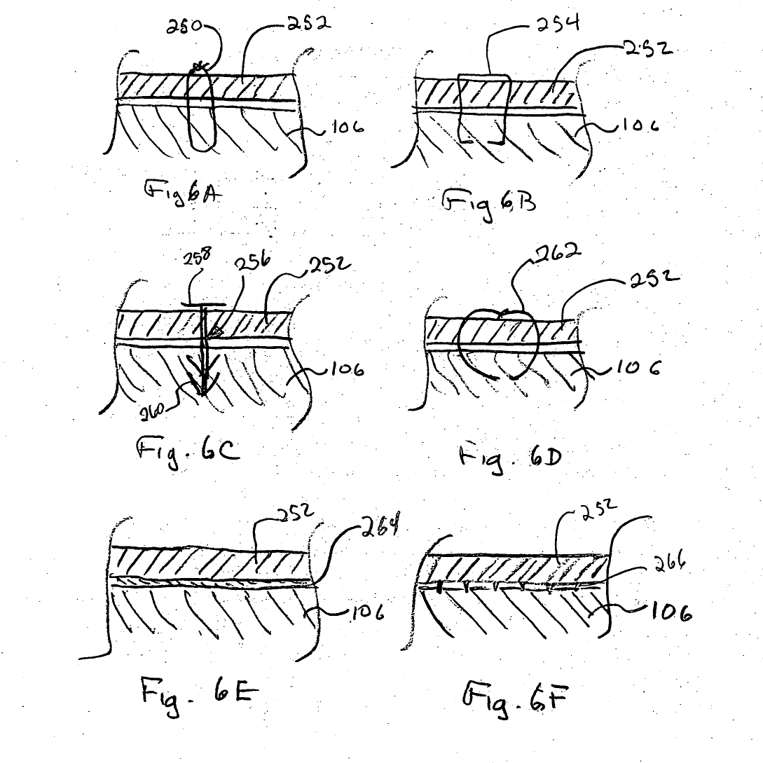

FIGS. 6A to 6F show examples of physical affixing components or adhesives that are especially suitable for fixing the upper section (202) to the pylorus or to the stomach wall.

FIG. 6A shows one variation of a suture fastener (250) used to affix the upper section (252) of the device (200) to the tissue of the pylorus (106). Several sutures (250) may be spaced about the upper section (252) to secure the upper section firmly to the pylorus (106).

Suture fasteners (250) may be introduced to the upper section (252) in a variety of ways after the upper section has been preliminarily situated upon the pylorus (106). U.S. Pat. No. 4,328,805, to Akopov et al, and published U.S. Pat. Appl. No. 2005/011967, to Reydel et al, describe devices suitable for introducing such sutures to join the upper section (252) to the tissue of the pylorus.

Suture fasteners (250) may be comprised of a variety of appropriate materials, e.g., biocompatible polymers and metals or alloys.

Appropriate biocompatible materials include natural materials, synthetic materials and combinations thereof. Natural or biological materials for use as sutures include relatively intact or cellular tissues as well as decellularized tissue. These tissues may be obtained from, for example, from connective tissues; tendons; ligaments, cartilage, and the like.

Natural tissues are derived from a particular animal species, typically mammalian, such as human, bovine, or porcine. These natural tissues generally include collagen-containing material. Appropriate tissues also include tissue equivalents such as tissue-engineered material involving a cell-repopulated matrix, which can be formed from a polymer or from a decellularized natural tissue.

Suitable synthetic materials include, for example, polymers, metals, alloys, and their mixtures. Pyrolytic carbon fiber may also be used. Appropriate metallic materials include metals and alloys based on titanium (such as nitinol, nickel titanium alloys, thermo-memory alloy materials), platinum, tantalum, nickel-chrome, or cobalt-chromium (such as Elgiloy® and Phynox®) and alloys such as various stainless steels, spring steel alloys, and the like.

Appropriate synthetic polymers include both resorbable and non-resorbable polymers. Non-resorbable polymers include polyamides (e.g., various Nylons), polyolefins such as polypropylene and polyethylenes, and polyfluorocarbons such as polytetrafluoroethylene (PTFE).

Suitable resorbable or biodegradable polymers include polyglycolide (PGA), polyglycolide copolymers, glycolide/lactide copolymers (PGA/PLA), glycolide/trimethylene carbonate copolymers (PGA/TMC), stereoisomers and copolymers of PLA, poly-L-lactide (PLLA), poly-D-lactide (PDLA), poly-DL-lactide (PDLLA), L-lactide/DL-lactide copolymers, L-lactide/D-lactide copolymers, copolymers of PLA, lactide/tetramethylene glycolide copolymers, lactide/trimethylene carbonate copolymers, lactide/δ-valerolactone copolymers, lactide/ε-caprolactone copolymers, polydepsipeptides (glycine-DL-lactide copolymer), PLA/ethylene oxide copolymers, asymmetrically 3,6-substituted poly-1,4-dioxane-2,4-diones, poly-β-hydroxybutyrate (PHBA), PHBA/β-hydroxyvalerate copolymers (PHBA/PHVA), poly-β-hydoxypropionate (PHPA), poly-β-dioxanone (PDS), poly-δ-valerolactone, poly-ε-caprolactone, methylmethacrylate-N-vinylpyrrolidone copolymers, polyesteramides, polyesters of oxalic acid, polydihydropyranes, polyalkyl-2-cyanoacrylates, polyurethanes (PU), polyvinyl alcohol (PVA), polypeptides, poly-β-maleic acid (PMLA), poly-β-alkanoic acids, polyethylene oxide (PEO), and chitin polymers.

Biological polymers may be naturally occurring or produced in vitro by, for example, fermentation and the like. Purified biological polymers may be appropriately formed into a substrate by techniques such as weaving, knitting, casting, molding, extrusion, or the like. Suitable biological polymers include collagen, elastin, silk, keratin, gelatin, polyamino acids, cat gut sutures, polysaccharides (e.g., cellulose and starch), and copolymers thereof.

FIG. 6B shows a staple fastener (254) affixing the upper section (252) to the pylorus (106). The staple fastener (254) may comprise a material having sufficient strength, malleability, and stiffness to be inserted through the upper section (252), into the pylorus (106), and to retain its shape after the insertion. Suitable materials include many of the polymeric and metallic materials listed just above, but stainless steels and NiTi alloys are especially suitable.

U.S. Pat. No. 5,725,554, to Simon et al, shows a stapler and staple suitable for introducing staples (254) as shown.

FIG. 6C shows a barbed brad-type fastener (256) having a large head (258) and barbs (260) that expand after piercing the upper section (252) and the pylorus (106).

FIG. 6D shows another variation of a staple fastener (262) piercing the upper section (252) and the pylorus (106). U.S. Pat. No. 6,773,440, to Gannoe et al, shows a device suitable for introducing such staples.

FIG. 6E shows an upper section (252) layer adhesively attached to the pylorus (106) by an adhesive layer (264). Suitable adhesives include cyanoacrylates such as butyl-2-cyanoacrylate, ethyl-2-cyanoacrylate, and octyl-2-cyanoacrylate; acrylic acid polymers and salts; fibrin glues such as mixtures of fibrinogen, thrombin, calcium chloride and factor VIII; cellulose derivatives such as carboxymethyl and hydroxypropyl methyl cellulose and their salts; derivatives of hydroxypropyl cellulose and methyl cellulose; a hydrogel comprising gelatin cross-linked with poly(L-glutamic acid) (PLGA); gelatin-resorcinol formaldehyde-glutaraldehyde; tragacanth, caraya, locust bean and other synthetic and natural gums such as algin, chitosan, starches, pectin, and naturally-occurring resins; polymers having suitable adhesive properties such as polyurethanes with amino groups, di- and tri-functional diols; polyvinyl acetates; polyamides; polyvinyl alcohols; polyvinyl pyrrolidone; polystyrene; polylactides; polylactones; block co-polymers including polyesters, polyamides, and polyurethanes; and their combinations and mixtures.

FIG. 6F shows the upper section (252) and the pylorus (106) joined by a plurality of short barbs (266) that extend from the upper section (252). The barbs (266) may be straight or curved.

FIG. 7 shows one variation of the upper section (266) where the section is a continuous membrane conforming in general shape to the pylorus (106). In the depicted variation, a plurality of sutures (268), such as depicted in FIG. 6A, is distributed about the membrane maintaining the shape of the upper section (266) against the pylorus. The upper section may comprise the polymeric materials discussed above.

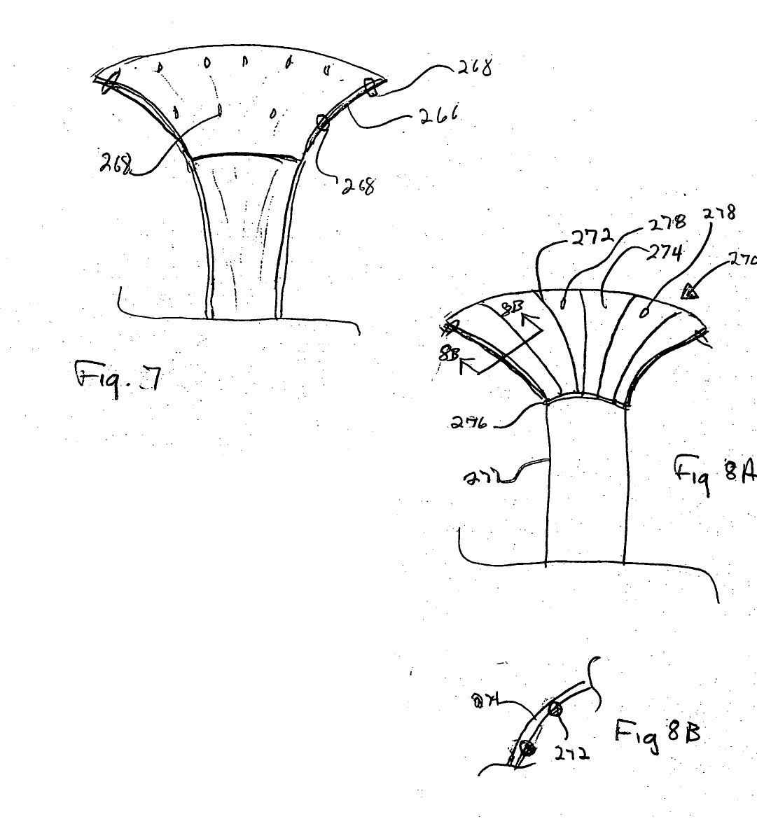

FIG. 8A shows a partial sectional view of another variation of an upper section (270) having stiffeners (272) to maintain the shape of the continuous membrane (274) against the surrounding pylorus. The stiffeners (272) may comprise one or more of the metallic or natural or synthetic polymeric materials discussed above. The stiffeners (272) provide longitudinal stiffening to the upper section (270) and to the component continuous membrane (274). The stiffeners (272) may be any of a wide variety of stiffnesses, ranging from quite stiff to soft—in the sense that the stiffener is only a bit stiffer than the continuous membrane in which it is situated. This variation of the upper section (270) is shown to be stabilized in position with sutures (276) although any appropriate fastener may be used. Indeed, the stiffeners (272) may be attached to a full or partial ring (276) in such a way that the stiffeners (272) provide a continuous pressure against the pylorus and maintain the device in place without fasteners, such as sutures (268).

FIG. 8B is a partial sectional view of the upper section (270) variation shown in FIG. 8A showing, in particular, the stiffeners (272) in position in the continuous membrane (274).

In the variation of the upper section (270) shown in FIGS. 8A and 8B, the stiffeners (272) and fasteners (276) may provide the sealing function discussed above. In other variations, an additional sealing structure or component may be necessary or desirable to provide any needed sealing.

The wall of the sleeve (277) passing through the pylorus may be thin and sufficiently flexible so that peristalsis is coupled to that sleeve's internal passageway. Such a sleeve (277) allows the pylorus to be used as a natural stoma in that the pylorus closes and then opens to allow passage of food when the muscles of the pylorus relax. That is to say that the sleeve (277) has enough wall flexibility or compliance to allow normal opening and closing of the pylorus to release and retain stomach contents and to allow drainage of chyme through the interior of the sleeve (277). The optional inclusion of folds, pleats, channels, or other structures in the sleeve (277) may be used to facilitate the collapse or expansion of the sleeve (277).

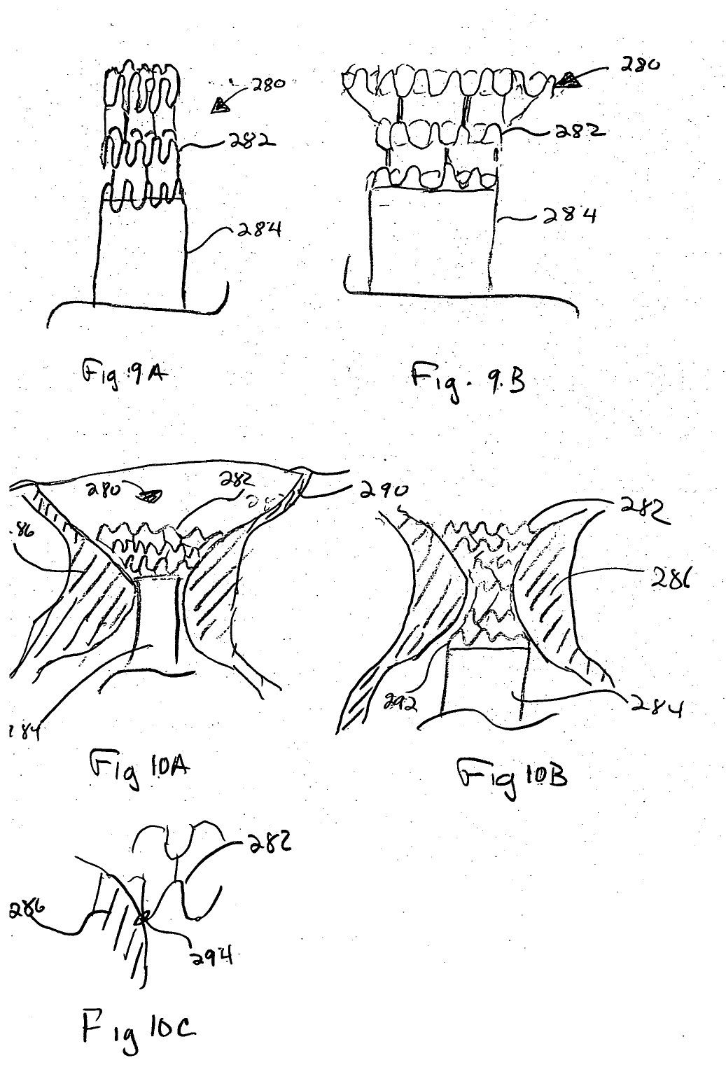

FIGS. 9A-9B show a variation of the upper section (280) in comprising a bare expandable stent-like structure (282) that may be affixed in the pylorus or proximal of the pylorus in the stomach. The stent-like structure (282) is attached to and supports the separator section (284). The separator section (284) is discussed below in more detail.

FIG. 9A shows the upper section (280) in a partially collapsed configuration as might be the situation during delivery of the device or during the expansion of the device after placement.

FIG. 9B shows the upper section (280) after expansion of the expandable stent-like structure (282) into the pylorus or the stomach to support the thus-implanted device. This stent-like structure (282) may be self-expanding or expandable using an expanding tool such as a balloon or other shaping tool.

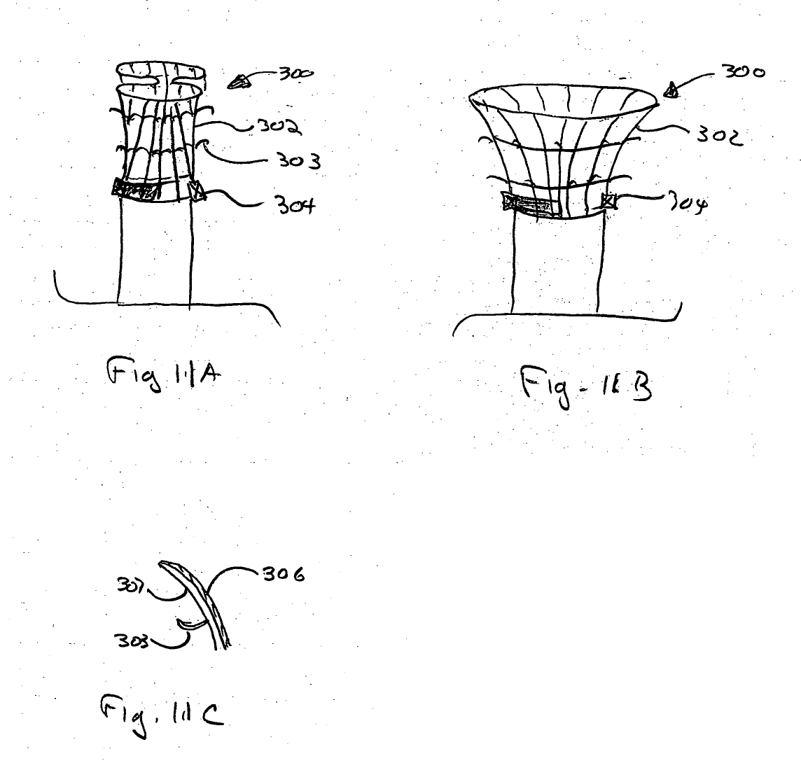

FIG. 10A shows implantation of the upper section (280) variation shown in FIGS. 9A and 9B into the proximal pylorus (286). The stent-like structure (282) may be extended into the distal stomach (290) if the designer so desires. In a variation of my device using such an upper section (280) design and placement, some other accommodation may be had for sealing, perhaps by its placement in the separator section (284).

FIG. 10B shows placement of the stent-like structure (282) into the pylorus (286) such that the open framework extends past the pylorus (286) and leaves open framework structure (292) in the duodenum. In this variation, some other accommodation must be had for the upper sealing function.

FIG. 10C shows optional securement of the stent-like structure (282) to the pylorus (286) with a fastener such as a suture (294). Such fixation may be optional if, e.g., the stent-like structure (282) is not self-expanding, the upper section (280) requires additional stabilization past that provided by the stent-like structure (282) itself.

The stent-like structure (282) and others described below may comprise any of the publicly-known materials used in vascular stents, e.g., various stainless steels, superelastic or shape-memory nitinols and other NiTi alloys, platinum-series metals and their alloys, gold and its alloys, polymeric materials, nickel-cobalt-chromium-molybdenum alloys having ultrahigh tensile strength, such as MP35N, etc. Mixtures of these materials are used in stents as are coatings of one on the other, e.g., gold as a plating layer upon nitinol or stainless steel to serve as a radiographic marker. Similar composite structures of the noted materials are known, e.g., partial coating of a metallic stent with polymeric materials to modify a bulk physical parameter such as stiffness, in a specific region of the stent.

The stent-like structure (282) may comprise one or more wires or ribbons making up the structure.

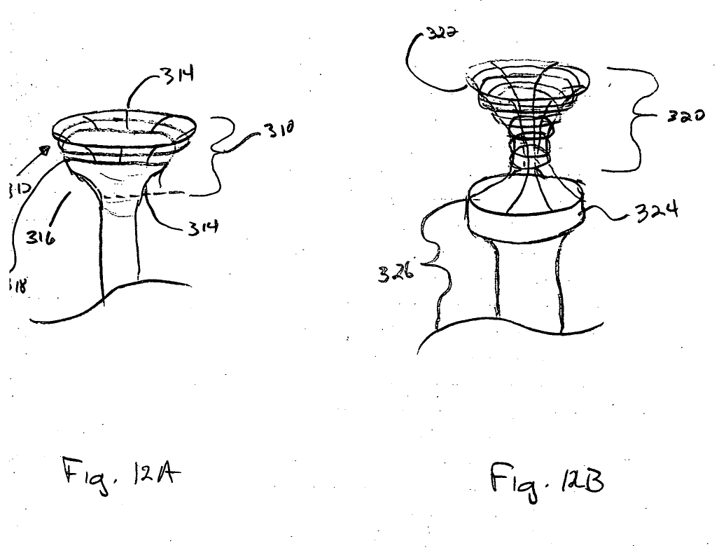

FIGS. 11A-11C show another variation of an upper structure (300) having a stent-like structure (302) with an open wire framework, optional continuous membrane, and barbs (303) that act as fasteners to the muscle of the pylorus.

In FIG. 11A, the stent-like structure (302) is depicted as being folded as would be the configuration during deployment of the device into the duodenum. Also shown in this variation is a component having a sealing function. This sealing component is shown in partial cross section and comprises an expandable foam ring (304). The expandable foam ring (304) comprises a foamed material, typically a closed cell biocompatible polymeric foam, that exerts a constant pressure against the pylorus after the device is implanted and therefore tends to hold the chyme exiting the stomach into the interior of the device.

FIG. 11B shows the stent-like structure (302) shown in FIG. 11B after its expansion during implantation. During the implantation step, which step may be carried out using an expander device such as a balloon or other functionally equivalent actor, if necessary, the barbs (302) are pressed into the muscle of the pylorus. The fastening barbs (303) are depicted as curved with the barbs (303) pointing distally to utilize the peristaltic action in continually securing the device to the pylorus. The barbs may be straight, include fish hook type barbs, or comprise other convenient shapes and may be oriented to enter the pyloric wall at an approximately 90° angle or other convenient angle. The barbs need not all be at the same angle.

FIG. 11C shows a close-up cross section of the stent-like structure (302), the continuous membrane (306), constituent ribs (307), and the fastening barbs (303) extending from the ribs.

FIGS. 12A and 12B show additional variations of the upper section, (310) and (330) respectively, utilizing stent-like structures, (312) and (332) respectively.

In FIG. 12A, the stent-like structure (312) has an open structure (314) that is to secure the device to the pylorus or stomach (or to both) using expansive pressure of the stent-like structure (312) upon the wall of the stomach or the pylorus or one or more other fasteners such as the fastening barbs (303) discussed elsewhere herein.

The stent-like structure (312) may extend down into the membrane portion (316) of the upper section (310) or may stop at the boundary (318) shown in the Figure. In this variation, the function of sealing the chyme inside of the device from the exterior of the device is borne by the membrane portion (316), with or without a separate seal structure. Details of acceptable seal structures are discussed elsewhere herein.

The variation of the upper section (320) shown in FIG. 12B comprises an open framework stent-like structure (322) that extends from the pylorus or distal stomach region down into the duodenum. In this variation, the upper section bears no sealing function but only supporting function. A sealing area (324) is shown as a component of the separator section (326).

The stent-like structure (312) shown in FIG. 12B may be self-adhering to the pylorus or stomach via pressure from the structure itself or may utilize fasteners such as the barbs (303) or the like shown elsewhere. The stiffness of the stent-like structure (312) may be selected to allow the pylorus to open and close in a normal fashion or to prevent the pylorus from closing.

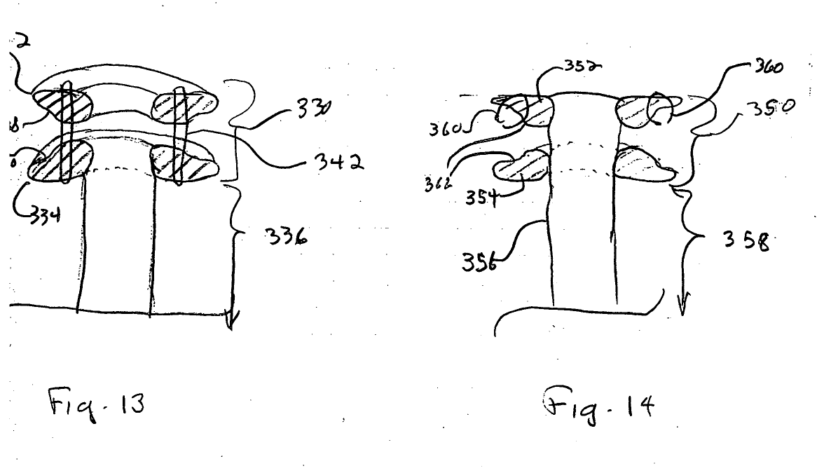

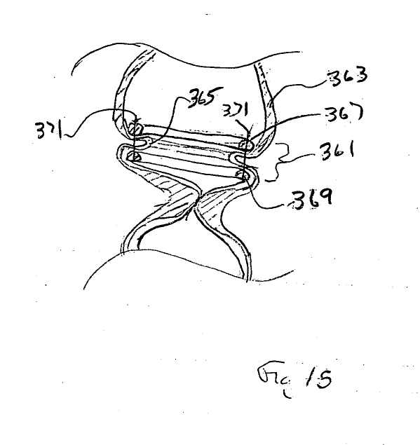

FIGS. 13 and 14 show variations of the upper section, (330) and (350) respectively, having sealing components proximal and distal of the pylorus.

In FIG. 13, the upper section (330) comprises an upper seal member (332) and a lower seal member (334) that cooperate to press against the pylorus and seal the exterior of the separator section (336) from chyme interior to that section (336). Typically, the upper seal member (332) and lower seal member (334) have some measure of compressibility, adequate to provide the sealing function. The outer periphery of the upper seal member (332) may include a groove (338) and the outer periphery of the lower seal member (334) may include a groove (340) to accept the pylorus. The upper seal member (332) and lower seal member (334) are joined to each other and to the pylorus via fasteners such as the sutures (342) depicted in the FIG. 13. Other functionally equivalent fasteners such as staples are also suitable.

In this variation, the lower seal member (334) is affixed to the separator section (336).

The variation of the upper section (350) shown in FIG. 14 also comprises upper seal member (352) and a lower seal member (354). The upper seal member (352) is affixed to the tubing (356) that extends from the upper section (350) down into and also forms a component of the separator section (358). The upper seal member (352) is also affixed to the pylorus or stomach by fasteners such as the removable staples (360) shown there. Closed staples, sutures, adhesives, and the like are also appropriate for such service.

The lower seal (354) may be affixed to the tubing (356) and, in such a variation, no fasteners need be included in the lower seal member (354). As is the case with the variation shown in FIG. 12A, the upper seal member (332) and lower seal member (334) may include grooves (362) to accommodate situating the upper section (350) about the pylorus.

FIG. 15 shows a variation of the upper section (361) that is fixed in position by plicating the stomach wall (363) by, e.g., suction and bracketing the so-formed fold (365) by an upper ring (367) and a lower ring (369). The upper ring (367) and lower ring (369) may be fixed in place by one or more fasteners (371) such as sutures, staples, etc. that may penetrate the plicated stomach wall. The fasteners (371) are shown to meet both the upper ring (a4) and lower ring (369) and the plicated stomach wall (365) but need not do so; the fasteners may penetrate only one of the upper ring (367) and lower ring (369) or may not penetrate the plicated stomach wall (365).

My device may have ancillary functions in addition to those discussed above with respect to its major function of maintaining separation of chyme from bile and pancreatic fluids through the duodenum and following sectors of the small intestine. For instance, the upper section may include one or more elements that are remain fixed in the stomach and effectively reduce the volume of the stomach. Other ancillary elements include one or more elements present in the stomach that mechanically interfere with the breakdown of the food in the stomach. Other ancillary elements include valving elements that release the contents of the stomach at selected intervals or orifice-style elements that, in effect, maintain the pylorus in a continuously open condition. These elements result in the food not being normally digested at the time of release from the stomach, prolonging digestion, or effectively decreasing the effectiveness of digestion.

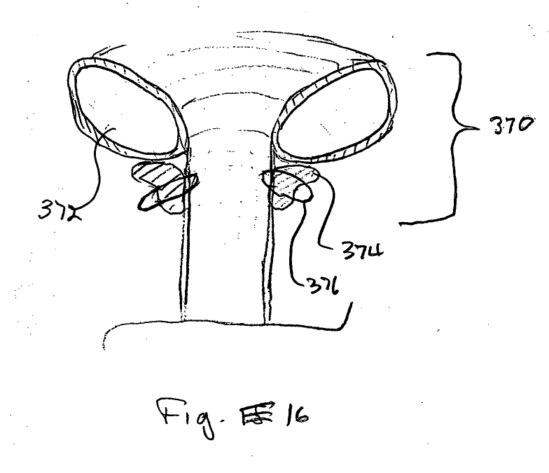

FIG. 16 shows a variation of the upper section (370) having an ancillary volume-occupying function. The upper section (370) includes a generally donut-shaped inflatable or inflated component (372) that is operable to occupy a volume in the stomach and further to assist in treating obesity. The inflatable component (372) additionally may be configured to provide both an anchoring or fixing function for the device and to provide a sealing function. An optional seal member (374) and optional fastening members (376), e.g., suture or staple, are also shown. The inflatable component (372) may be manually inflated or self-inflating, as desired, with a gas or a liquid.

FIGS. 17A and 17B show, respectively, a perspective view and a side view of another variation of an upper section (380) having ancillary volume-filling inflatable components (382) attached to the membrane (384).

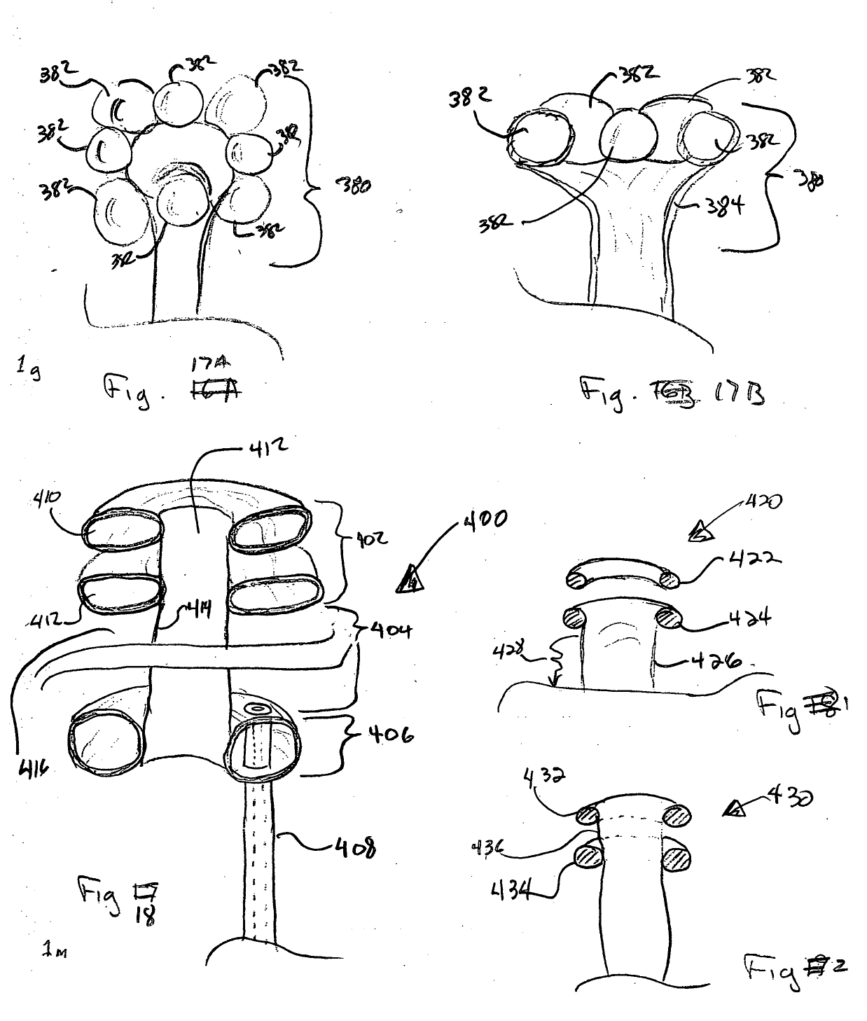

FIG. 18 shows a side, cross-section view of my device (400) having an upper section (402), a separator section (404), a lower seal section (406), and a conduit section (408). Of special interest for discussion here are the inflatable upper seal member (410) and the inflatable lower seal member (412) that lie adjacent the pylorus and are operable to affix the device (400) in place and to seal the interior passageway (414) from the exterior volume (416) formed by the lower seal member (412), the lower seal section (406), the separator section wall (414), and (after implantation) the wall of the duodenum.

Also shown in FIG. 18 is an inflatable lower seal section (406), discussed in isolation below.

As noted above and also discussed in more detail below, the conduit section (408) is in fluid communication with the exterior volume (416) and is operable to pass bile and pancreatic fluids distally in the small intestine.

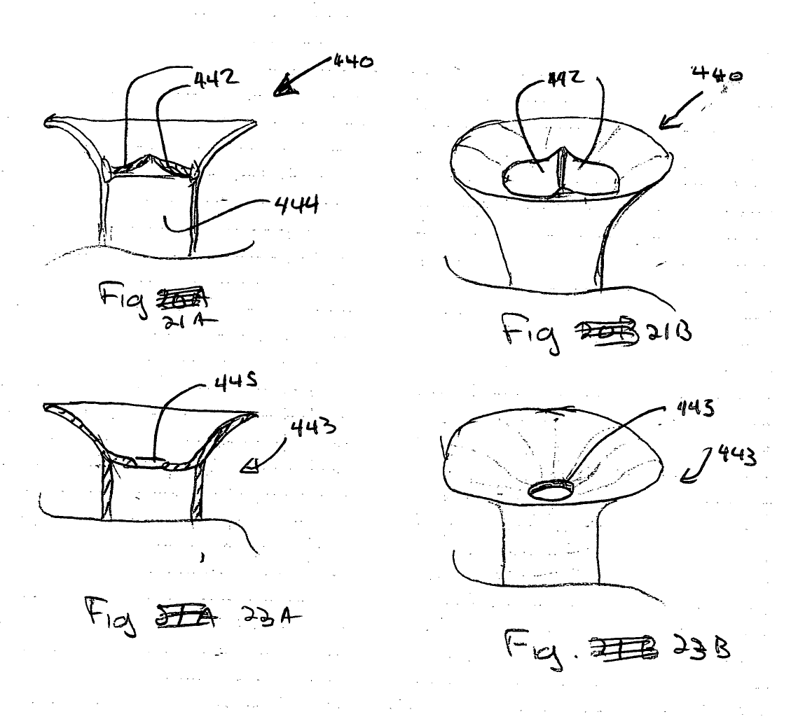

FIGS. 19 and 20 show variations of the upper section, (420) and (439) respectively, utilizing magnetic rings to fix the device in place.

FIG. 19 shows an upper magnetic ring (422) that is to be situated proximally of the pylorus and a lower magnetic ring (424) that is to be located distally of the pylorus. The lower magnetic ring (424) is attached to the wall (426) of the separator section (428) and provides sealing after implantation. The upper magnetic ring (422) and the lower magnetic ring (424) magnetically attract to couple and form a seal and joint with the pylorus.

FIG. 20 also shows an upper magnetic ring (432) and a lower magnetic ring (434) that magnetically cooperate and attract to form a seal and affix the device in place about the pylorus. In this variation, both the upper magnetic ring (432) and lower magnetic ring (434) are attached to wall (436).

The variations shown in the following Figures show an upper section that is implanted at the pyloric valve or sphincter. This variation includes a valving mechanism that generally causes the pylorus to stay open during the period of time when food is present in the stomach and thereby cause rapid passage of consumed food into the duodenum and yet to prevent retrograde flow of duodenal contents—and specifically bile—back into the stomach. This latter function prevents stomach ulcers and biliary damage to the gastric mucosa.

FIGS. 21A and 21B show, respectively, side cross-section and perspective views of another variation of an upper section (440) having a pair of biased valve leaves (442) that stay closed until a (usually quite small) design pressure is imposed upon the valve leaves (442) by the presence of chyme. This feature provides an ancillary function to my device by both delaying passage of chyme into the duodenum until the pressure of the stomach contents reaches the design limit and opening (and closing) quickly when the chyme pressure in the stomach rises and falls. This function may be used to aid in the treatment of obesity.

After passage of chyme into the passageway (444) below the valve leaves (442), the biased valve leaves (442) return to the closed position.

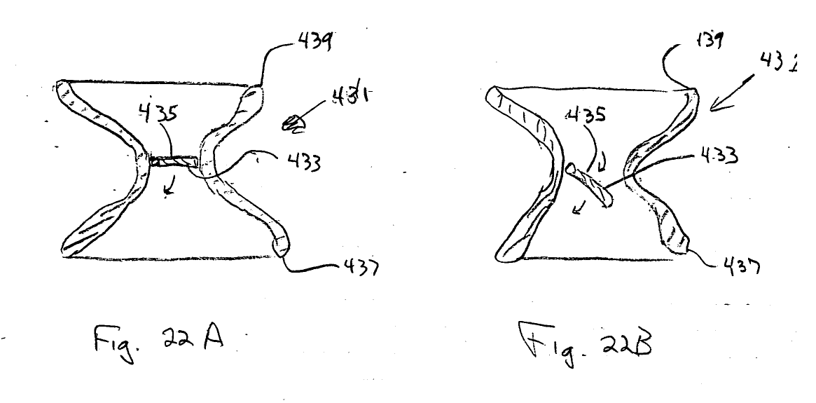

FIGS. 22A and 22B show another variation of an upper section (431) having a flap valve component (433), respectively, with the flap valve (433) closed and retaining contents in the stomach and with the flap valve (433) open allowing the contents of the stomach to pass into the duodenum. The flap valve (433) is spring biased to remain in the closed condition shown in FIG. 22A until the pressure on the upper surface (435) of the flap valve (433) reaches a design limit and opens as shown in FIG. 22B. The flap valve (433) closes after the chyme has passed into the duodenum.

Other valving variations include rotating door valves, funnel valves, and the like are also suitable if they meet the functional requirements discussed here.

The opposing ends of the valved variations of the upper sections discussed just above (i.e., (441) in FIGS. 21A and 21B and (437) and (439) in FIGS. 22A and 22B) typically have a diameter larger than the largest diameter of the pylorus opening. This end diameter allows the upper sections—(440) in FIGS. 21A and 21B and (431) in FIGS. 22A and 22B—to remain affixed in position. Appropriate fasteners may obviously be utilized to assist in maintaining the upper sections in position if so desired.

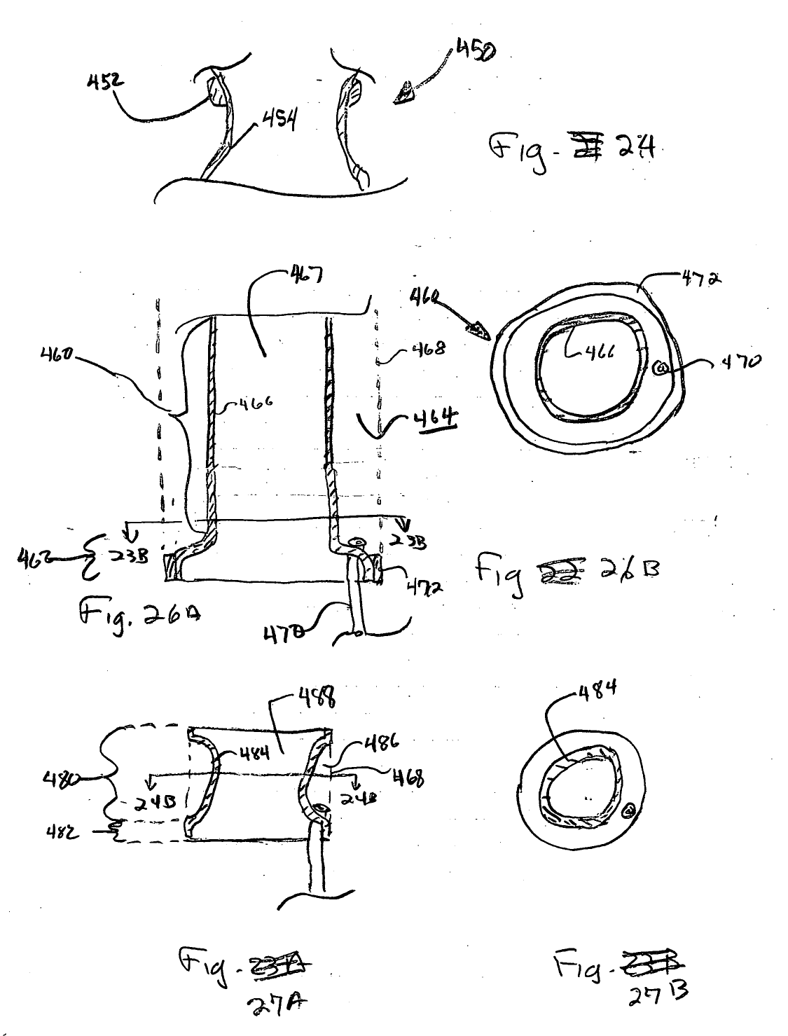

FIGS. 23A and 23B show, respectively, side cross-section and perspective views of another variation of an upper section (443) having an orifice (445) with a size selected to provide a continuing flow of chyme, the flow dependent principally upon the pressure in the stomach and the viscosity and solids content of the chyme.

FIG. 24 shows a side cross-section of an upper section (450) having a circular seal (452) comprising a compressible, resilient, polymeric foam that seals the upper section wall (454) against the pylorus. Such a foam would typically be a closed cell, biocompatible material suitable for providing the sealing function discussed elsewhere.

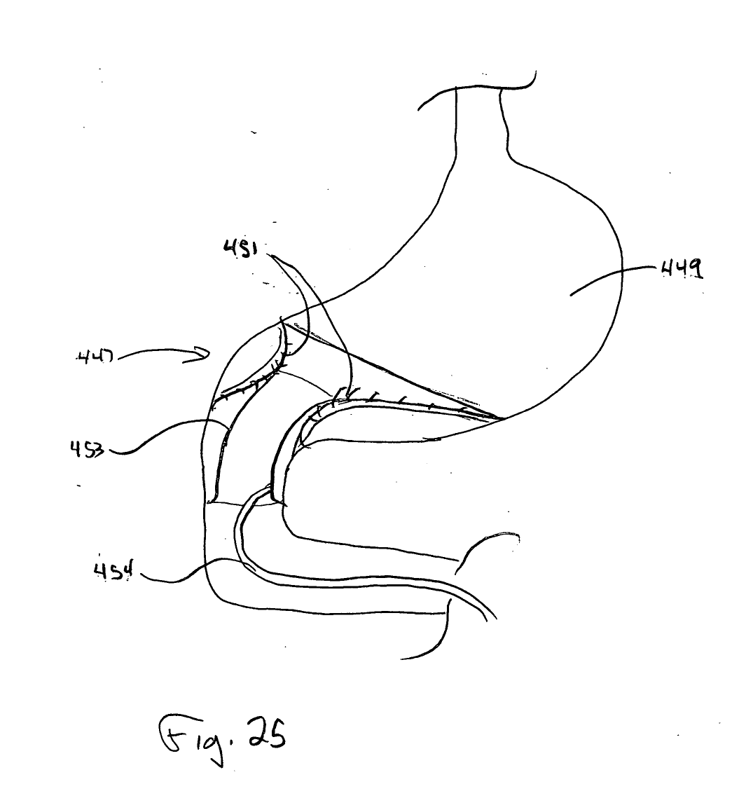

FIG. 25 shows an upper section (447) having an ancillary function, that of reducing the volume of the stomach (449) by stapling or suturing (451) the stomach wall. Those fasteners may also serve to provide fixation to the upper section (447). Also shown is the separator section (453) and a conduit member (454).

Generally speaking, the upper section variations of my device described herein may be independently attached to any of the variations of the separator section described here providing that the various functions described here are also carried out in the resulting combination.

### Separator Section

As described elsewhere, the separator section carries out the major functions of collecting bile and pancreatic fluids for delivery to the conduit section—the structure of which conduit section is discussed below—and maintaining separation of those fluids from chyme until that delivery. The separator section may include seals to provide appropriate separation or may cooperate with other sections, e.g., upper section, lower sealing section, for such sealing.

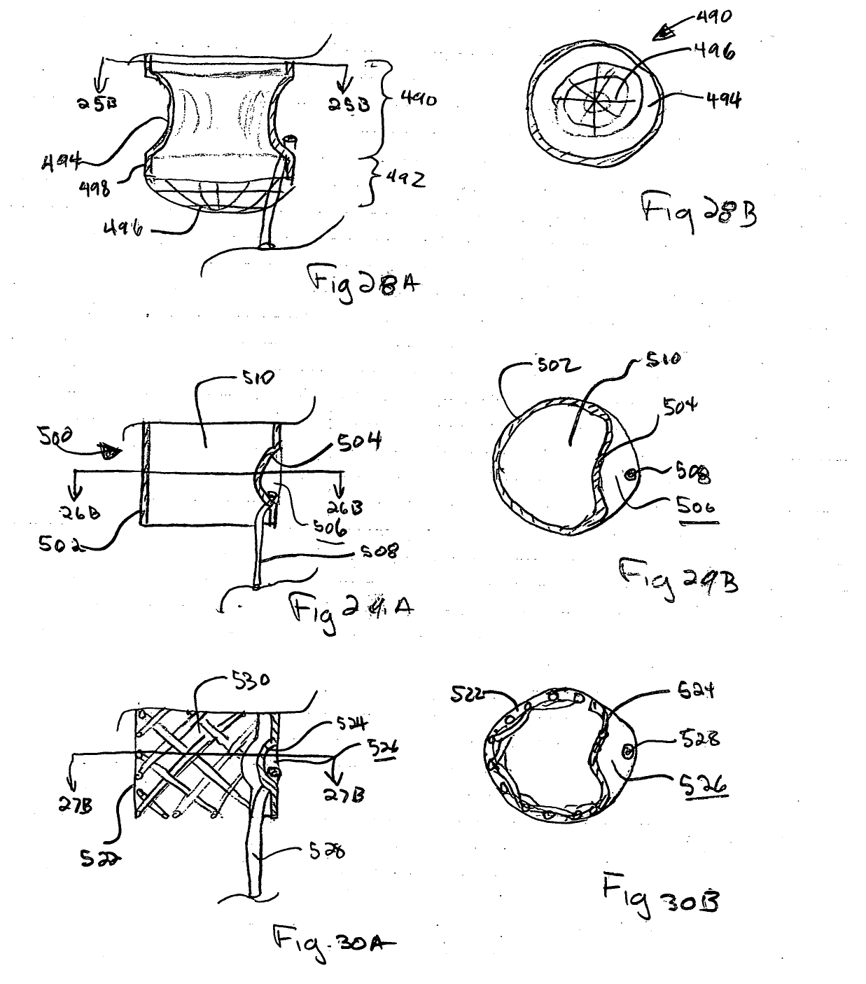

FIGS. 26A and 26B show, respectively, a side, cross-section view and a top, cross-section view of one variation of a separator section (460). The Figures also show the relationship of the separator section (460) to a lower sealing section (462). The separator section (460) depicted in FIGS. 26A and 26B comprises a substantially cylindrical wall (466), the exterior of which forms an annular volume (464) with the duodenal wall (468). The interior of wall (466) defines a passageway (467) for passage of chyme from the stomach. The stream containing bile and pancreatic fluids exiting the Ampulla of Vater enter that annular volume (464) and pass to the conduit section (470).

An independent seal (472) is shown in the lower seal section (462) that cooperates with the wall (466) of the separator section (460) to define the exterior annular volume (464).

FIGS. 27A and 27B show, respectively, a side, cross-section view and a top, cross-section view of one variation of a separator section (480) and its relationship to a lower sealing section (482). The separator section (480) comprises a substantially circular wall (484), the exterior of which forms an annular volume (486) with the duodenal wall (468). The interior surface of wall (484) defines the through-passageway (488) for chyme. The stream containing bile and pancreatic fluids exiting the Ampulla of Vater enters that annular volume (486).

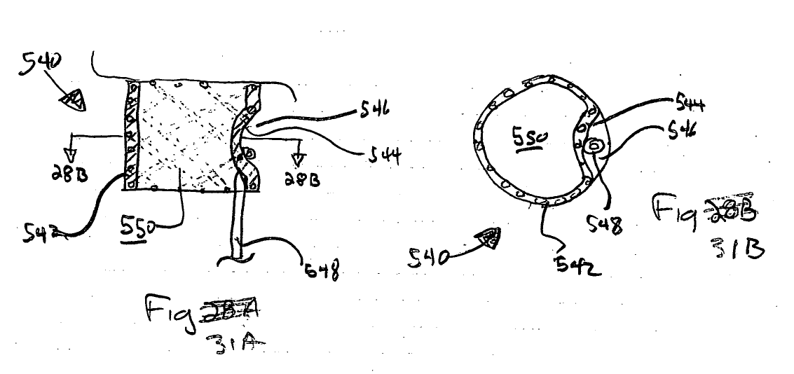

FIGS. 28A and 28B show, respectively, a side, cross-section view and a top, cross-section view of another variation of a separator section (490) and its relationship to a lower sealing section (492). The shape of the wall (494) of the separator section (490) is the same as that in FIG. 27A. In this variation, the lower sealing section (492) includes a stretcher component (496) that maintains the lower sealing section (492) against the wall of the duodenum. The stretcher component (496) comprises a number of diametrically situated, springy wires or ribbons that press the wall portion (498) of the lower sealing section (492) against the wall of the duodenum.

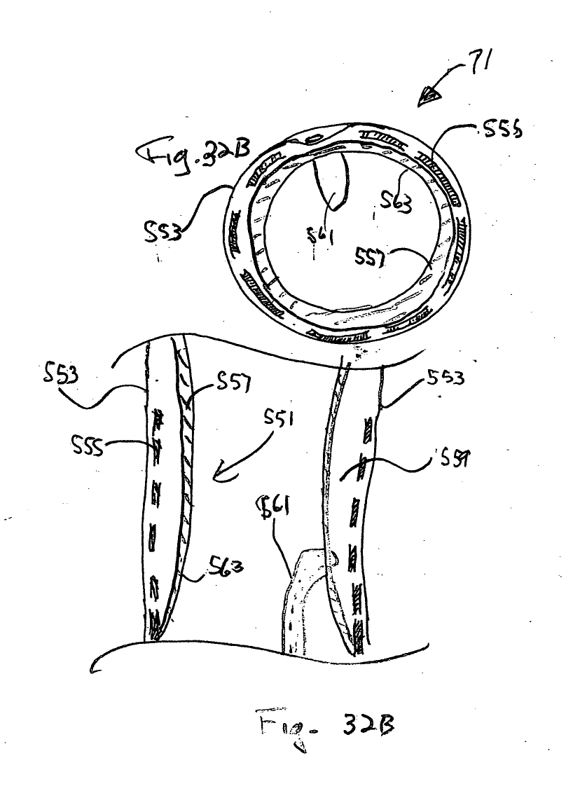

FIGS. 29A and 29B show, respectively, a side, cross-section view and a top, cross-section view of another variation of a separator section (500). The shape of the wall (502) of the separator section (500) is generally cylindrical with an inwardly extending blister (504). The blister (504) defines a volume (506) that is situated in the duodenum to enclose the Ampulla of Vater and accept the stream containing bile and pancreatic fluids. The blister volume (506) is in fluid communication with the conduit section (508). The interior of the wall (502) defines a chyme passageway (510).

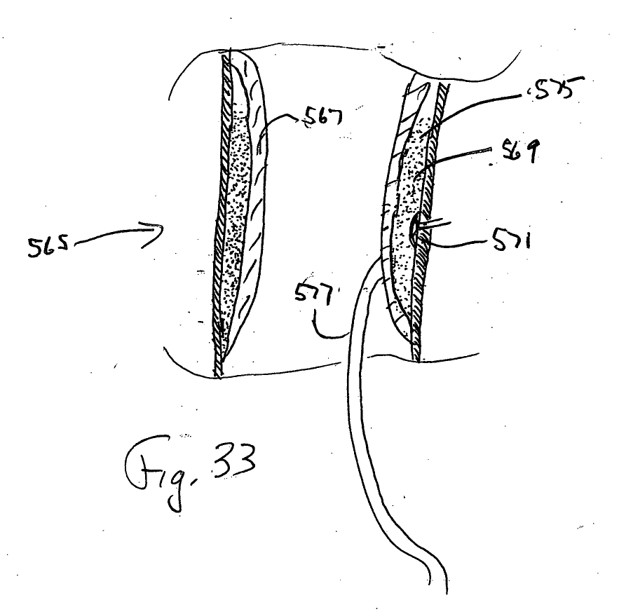

FIGS. 30A and 30B show, respectively, a side, cross-section view and a top, cross-section view of another separator section (520) variation. The wall (522) comprises a stent-like structure that supports a separate membrane forming a blister (524). The shape of the wall (522) of the separator section (520) is generally cylindrical excepting the separate, inwardly extending blister (524). The blister (524) is supported by the stent-like structure and defines a volume (526) that is situated in the duodenum to enclose the Ampulla of Vater and accept the stream containing bile and pancreatic fluids. The blister volume (526) is in fluid communication with the conduit section (528). The interior of the wall (522) defines a chyme passageway (530).

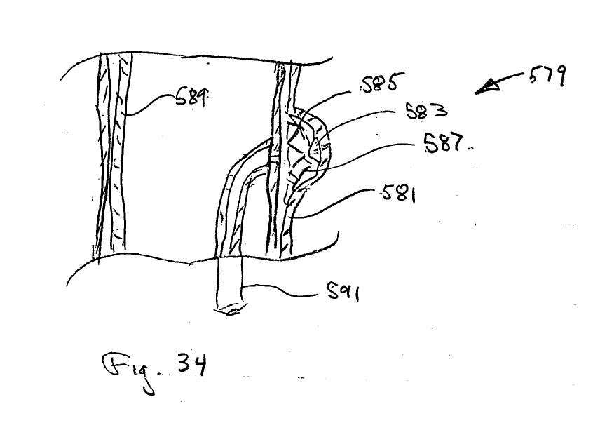

FIGS. 31A and 31B show, respectively, a side, cross-section view and a top, cross-section view of another separator section (540) variation. The wall (542) comprises a stent-like structure enclosed within a polymeric membrane and is generally cylindrical excepting the separate, inwardly extending blister (544). The blister (544) defines a volume (546) that is situated in the duodenum to enclose the Ampulla of Vater and accept the stream containing bile and pancreatic fluids. The blister volume (546) is in fluid communication with the conduit section (548). The interior of the wall (542) defines a chyme passageway (550).

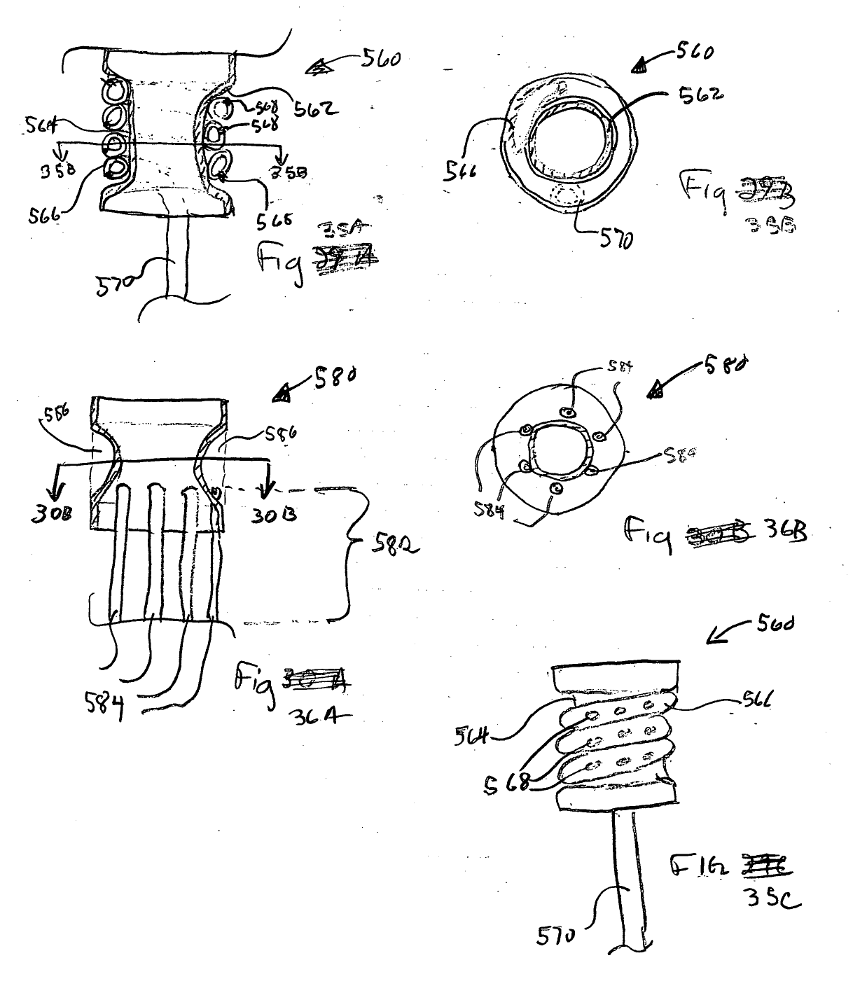

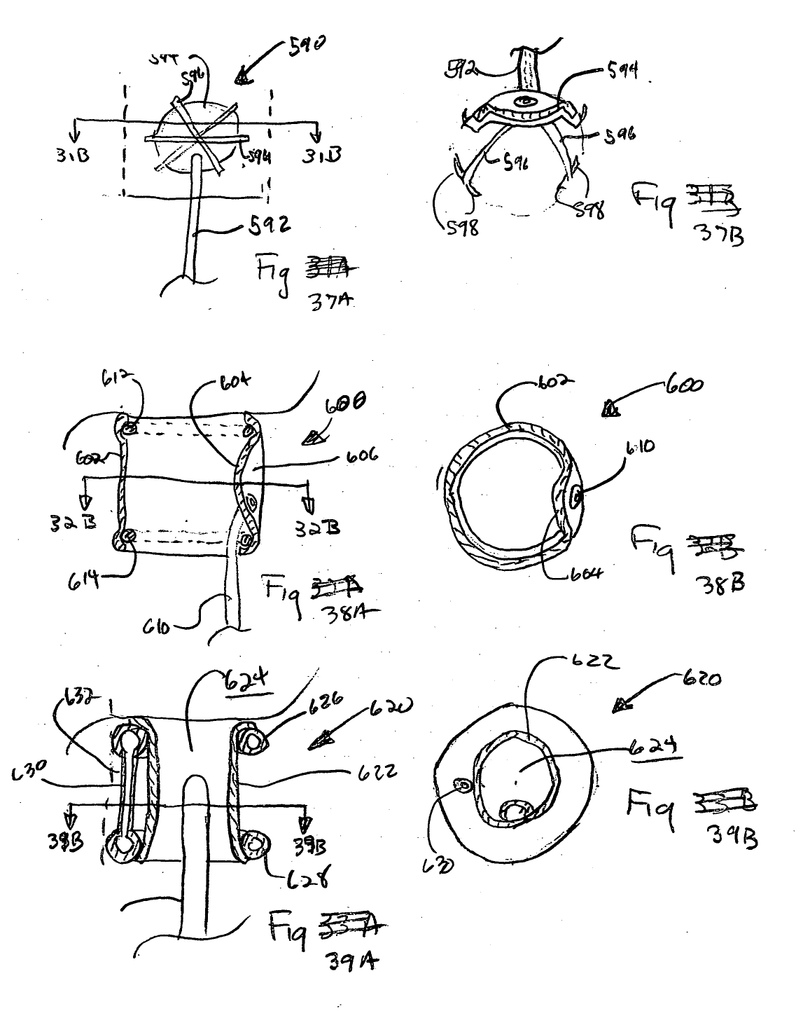

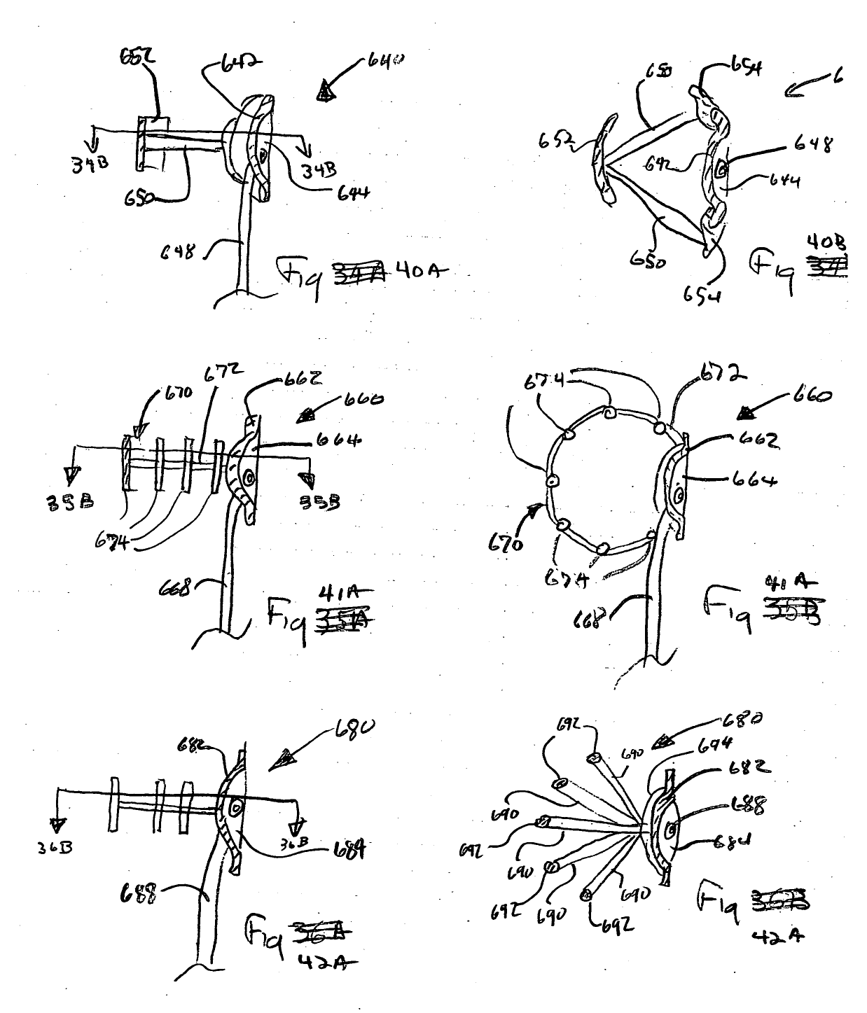

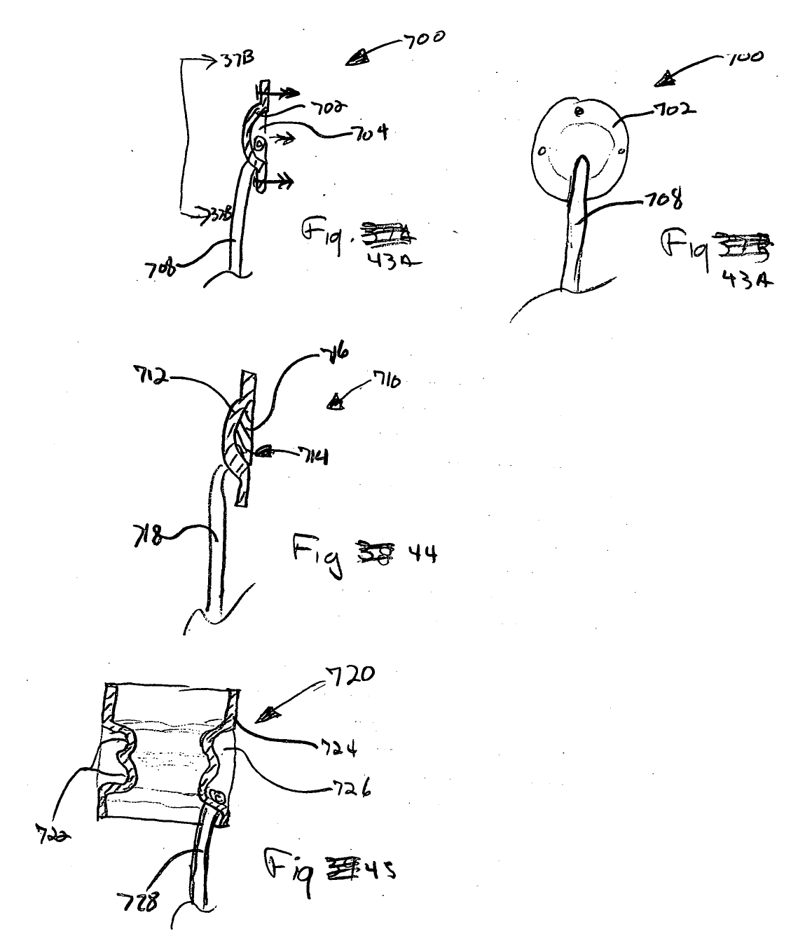

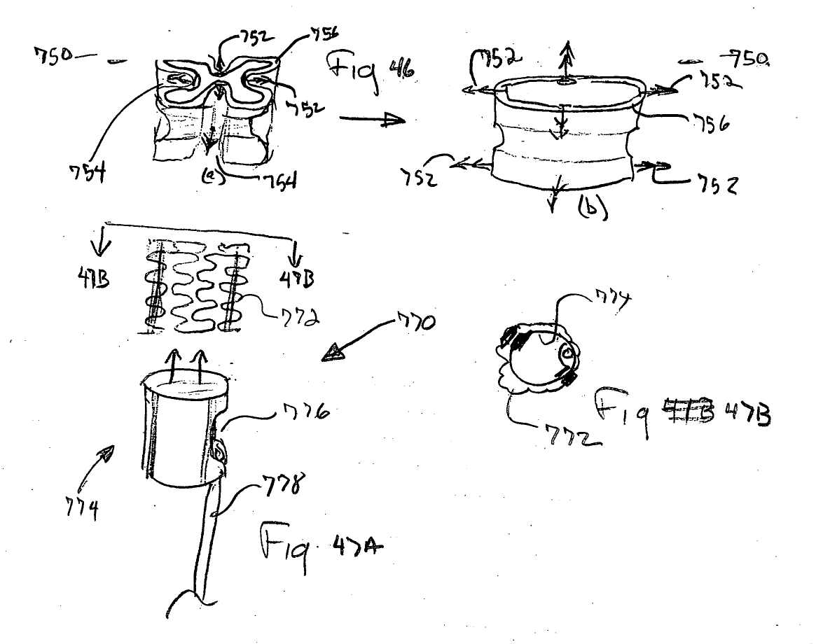

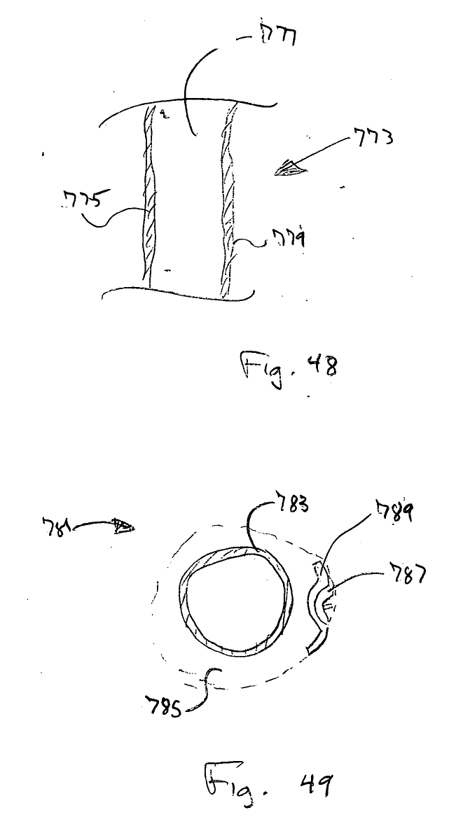

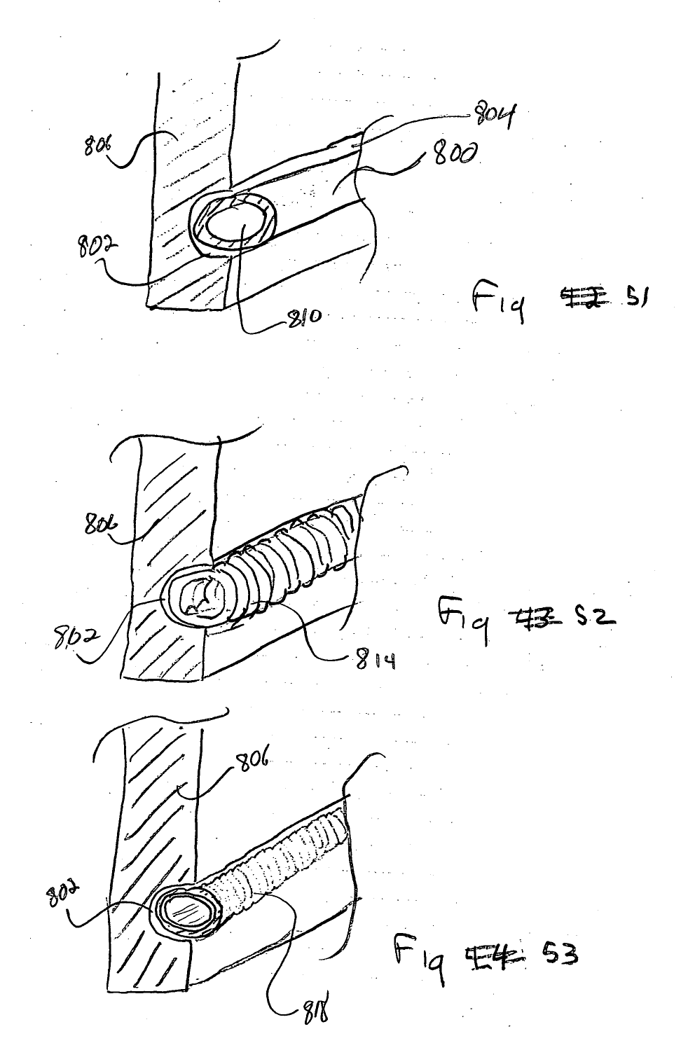

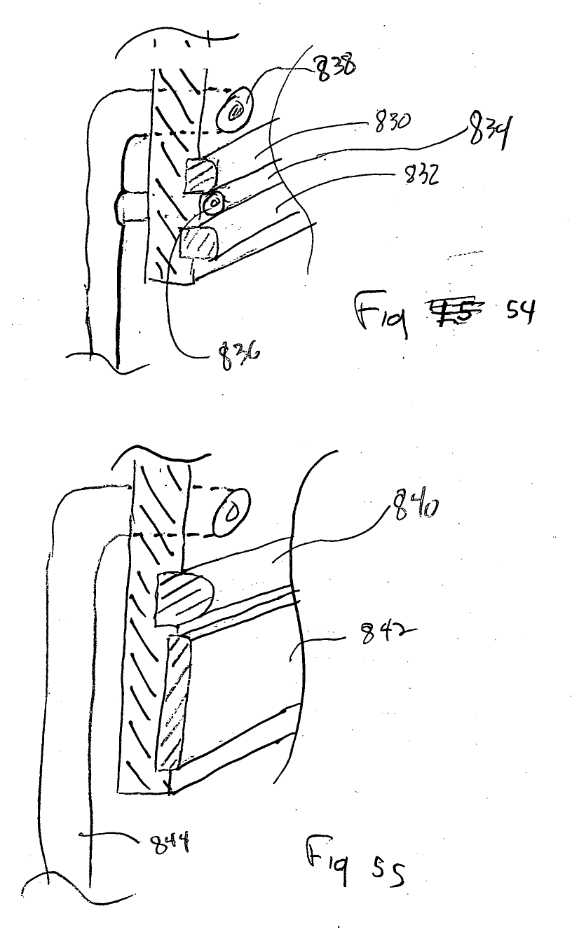

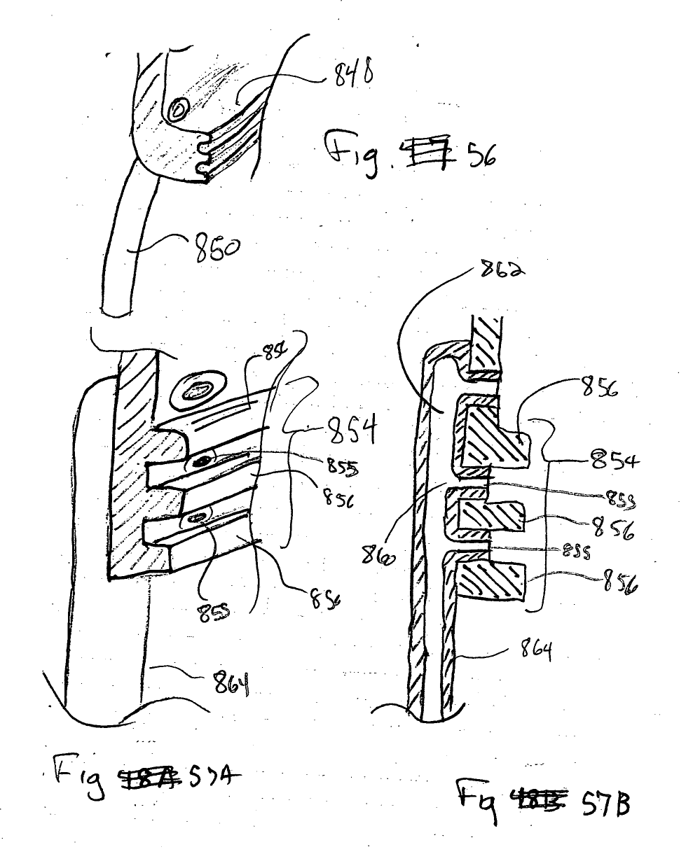

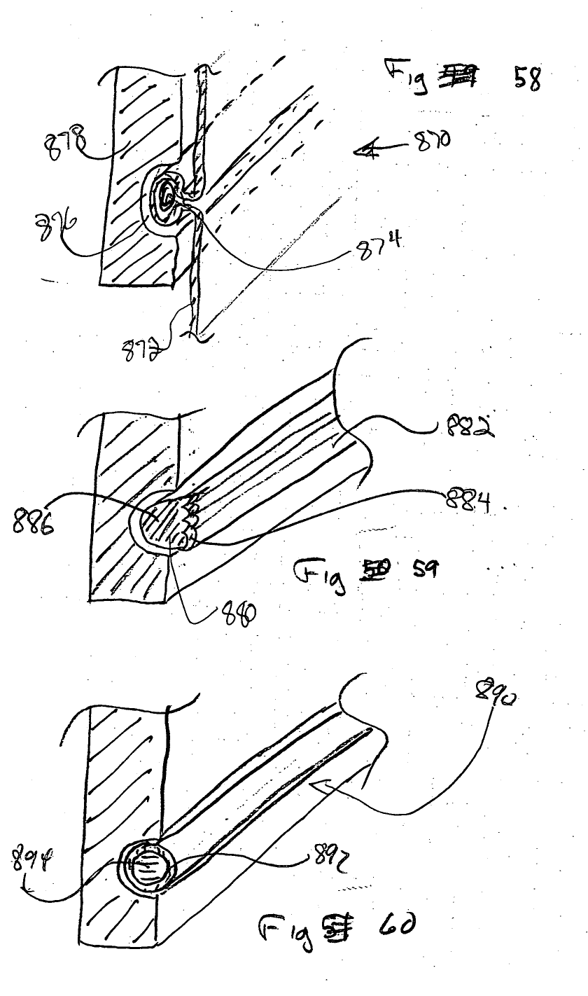

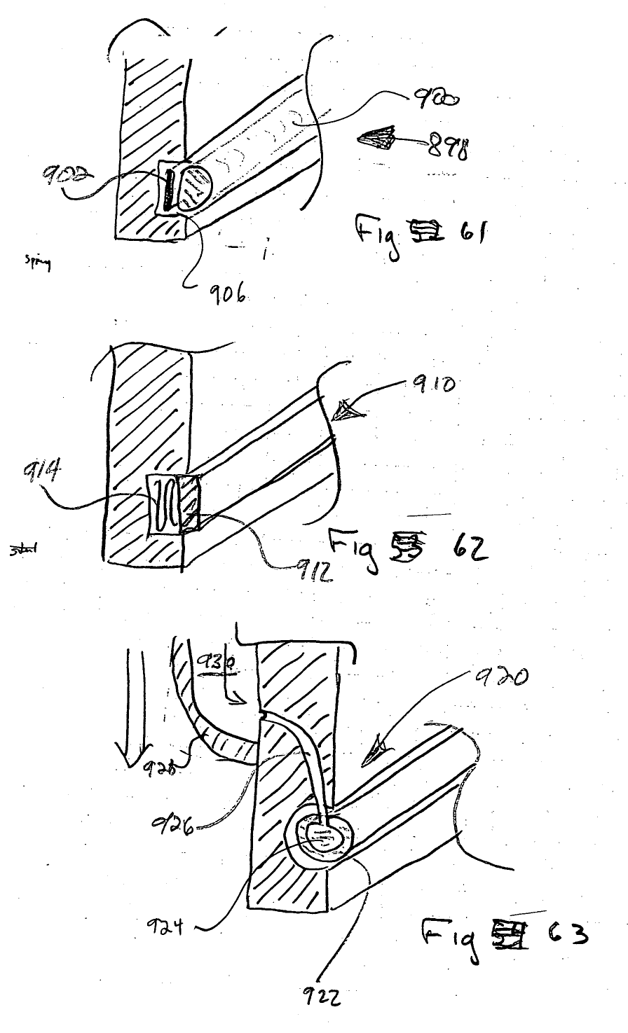

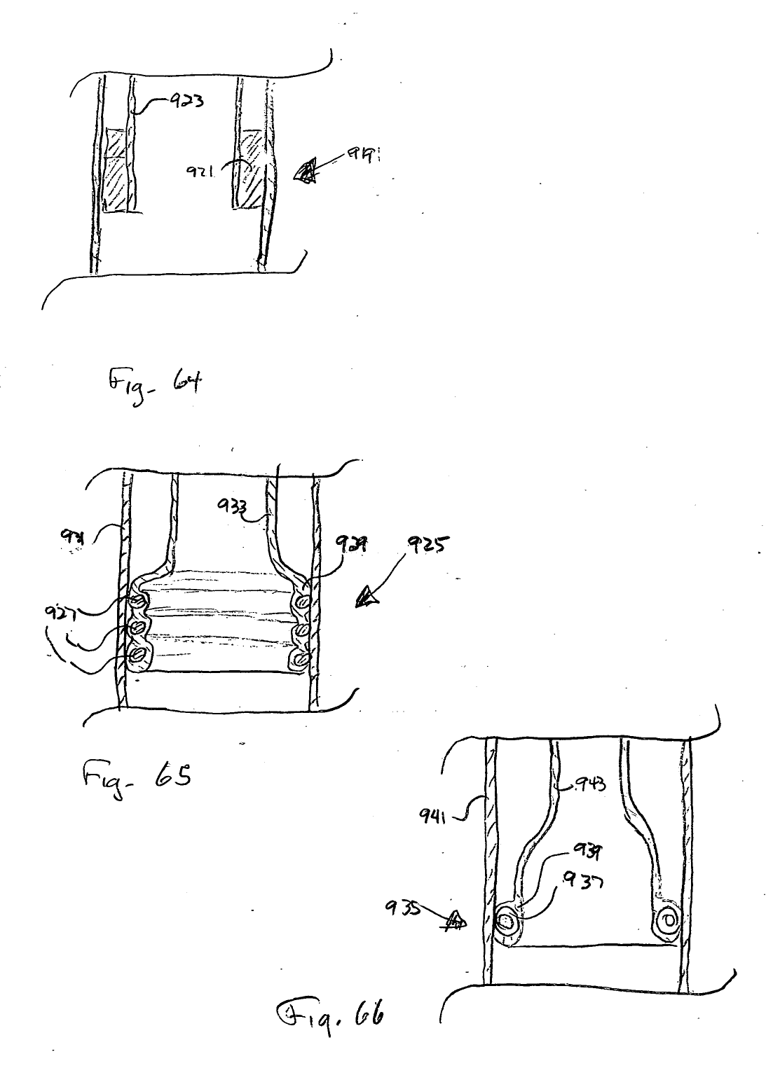

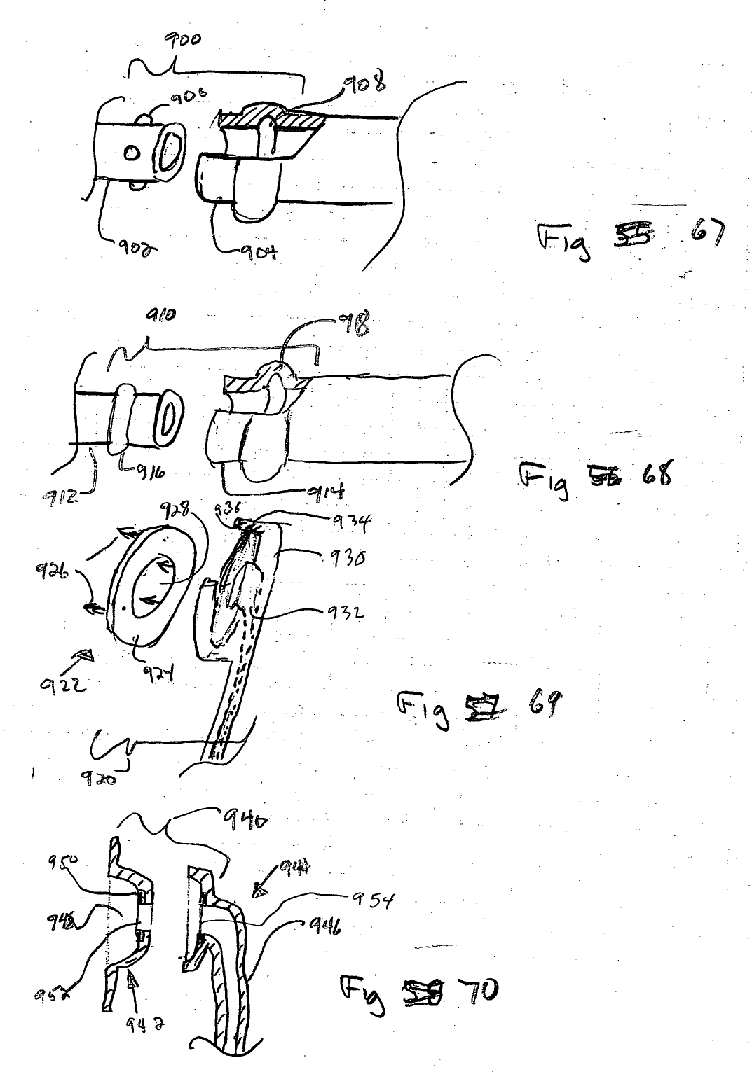

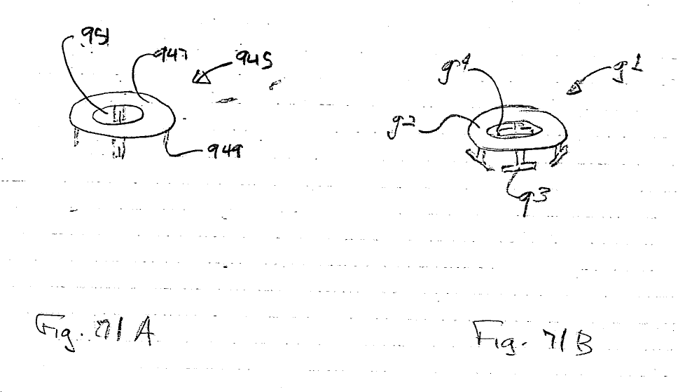

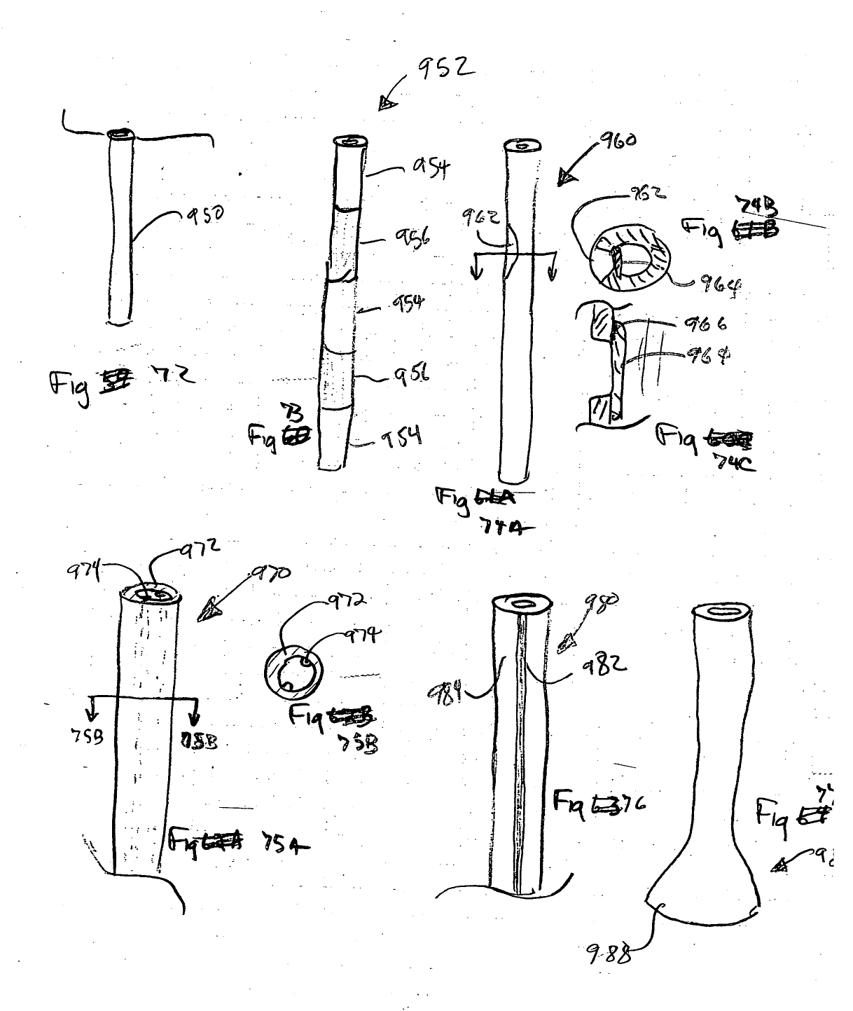

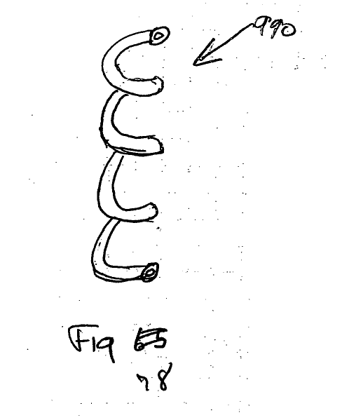

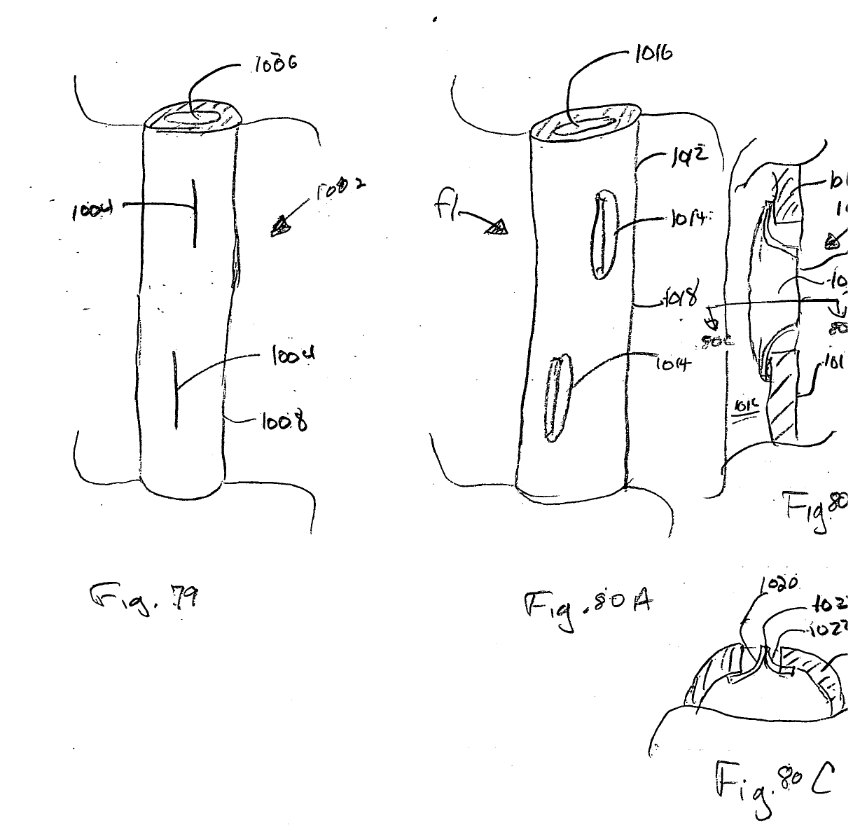

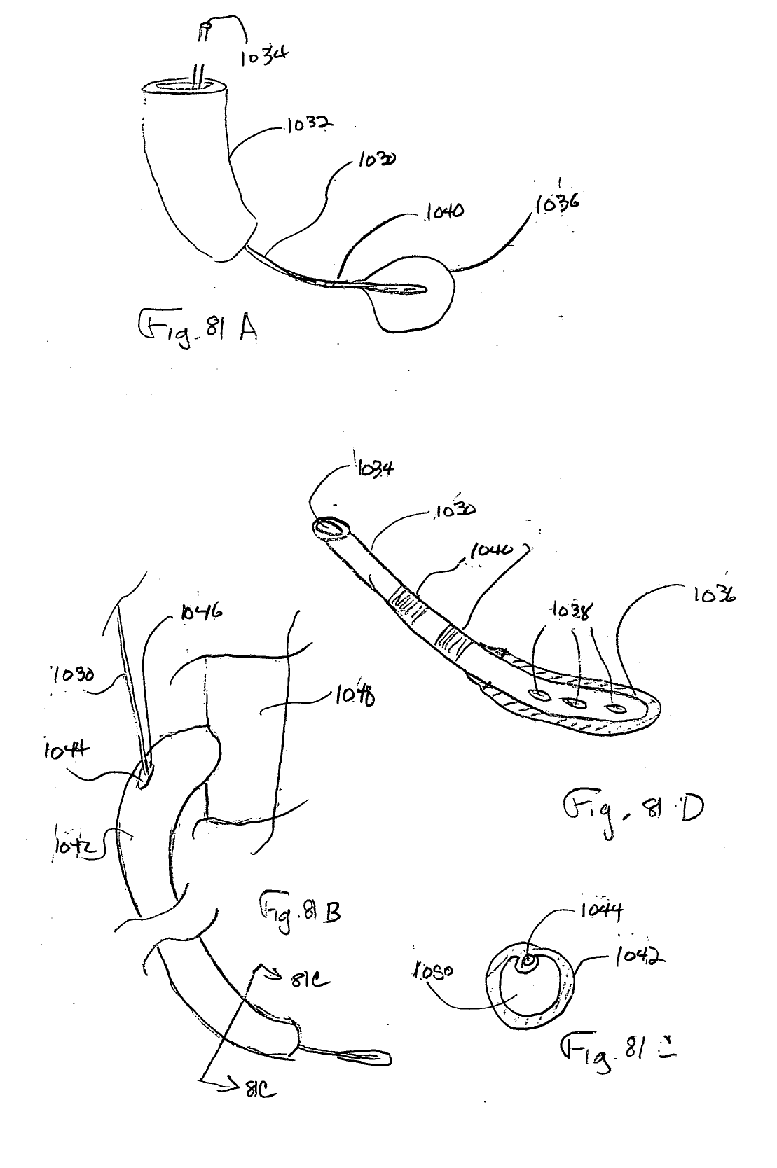

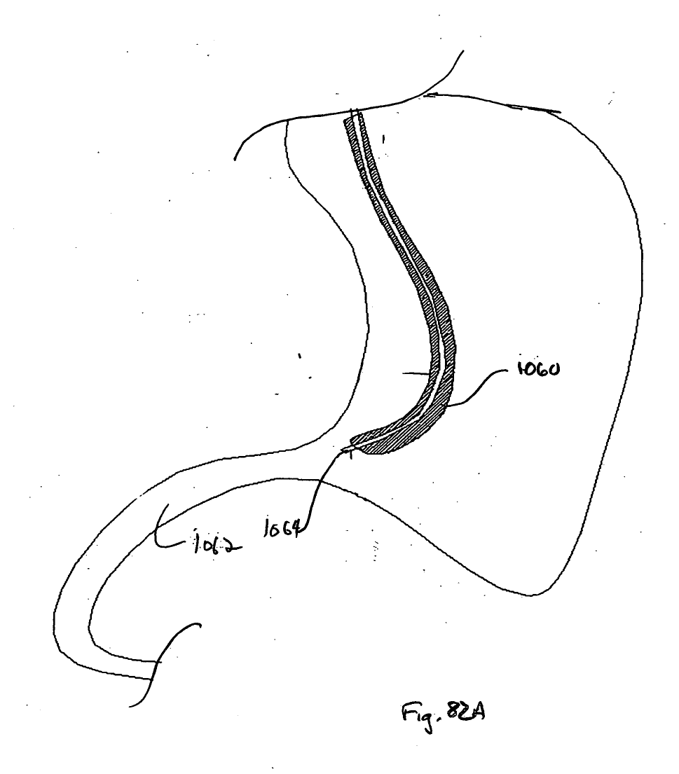

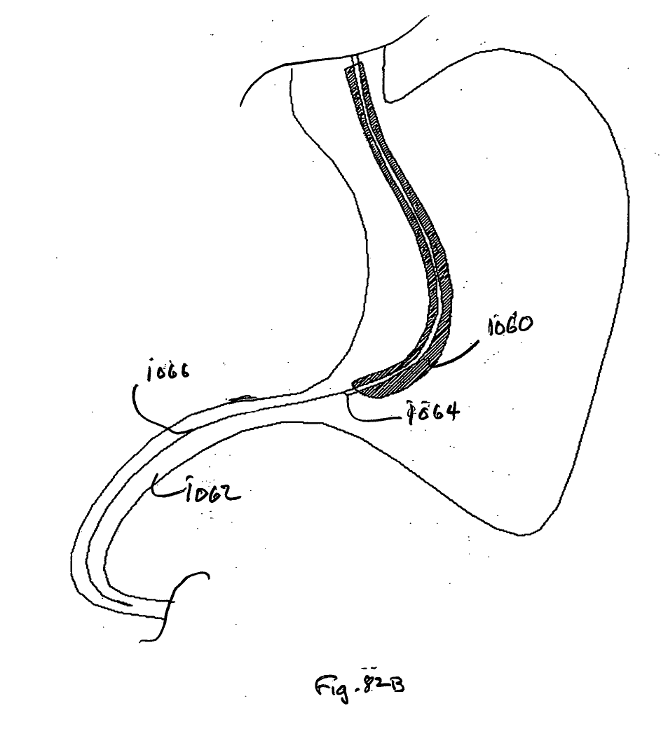

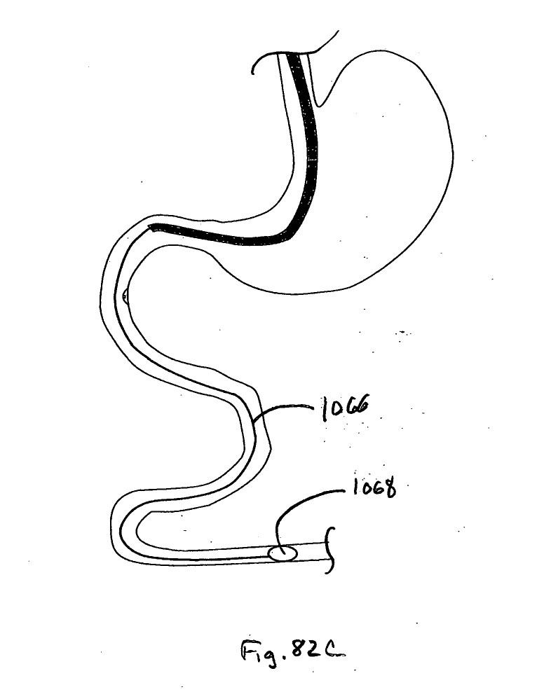

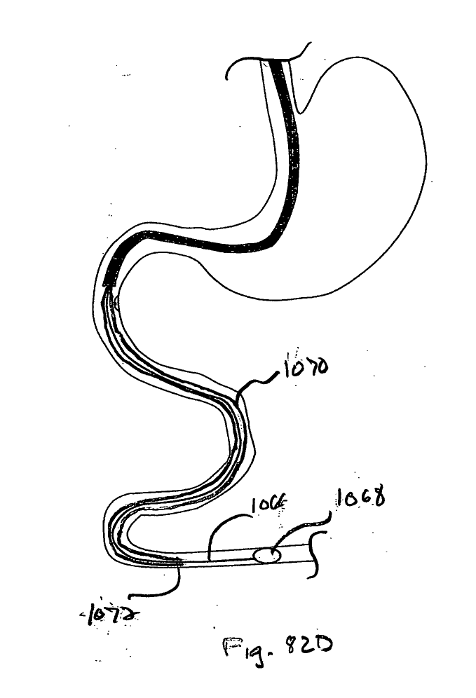

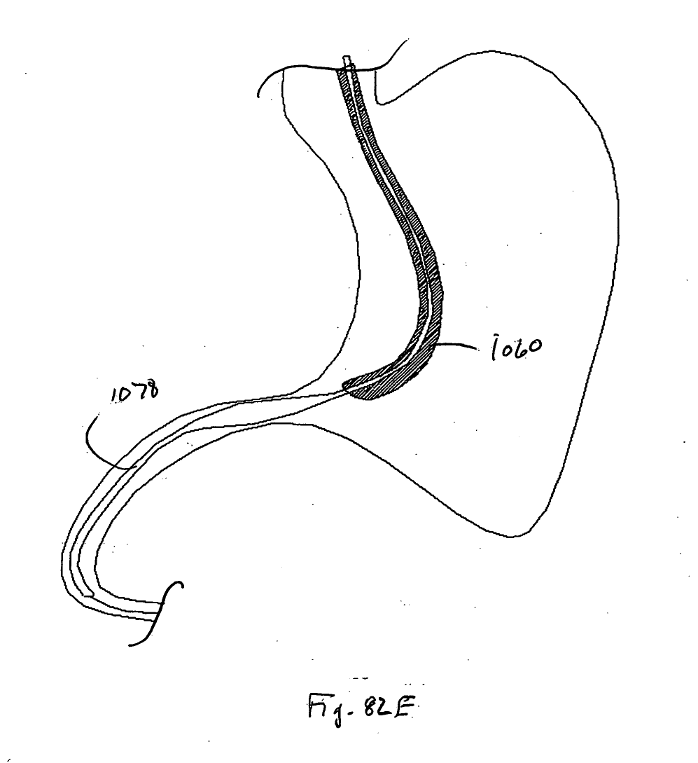

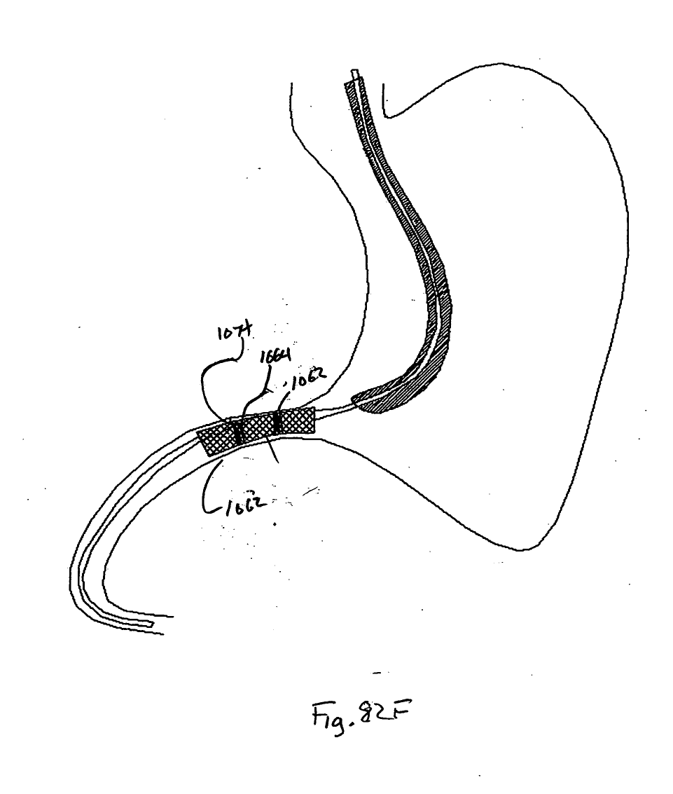

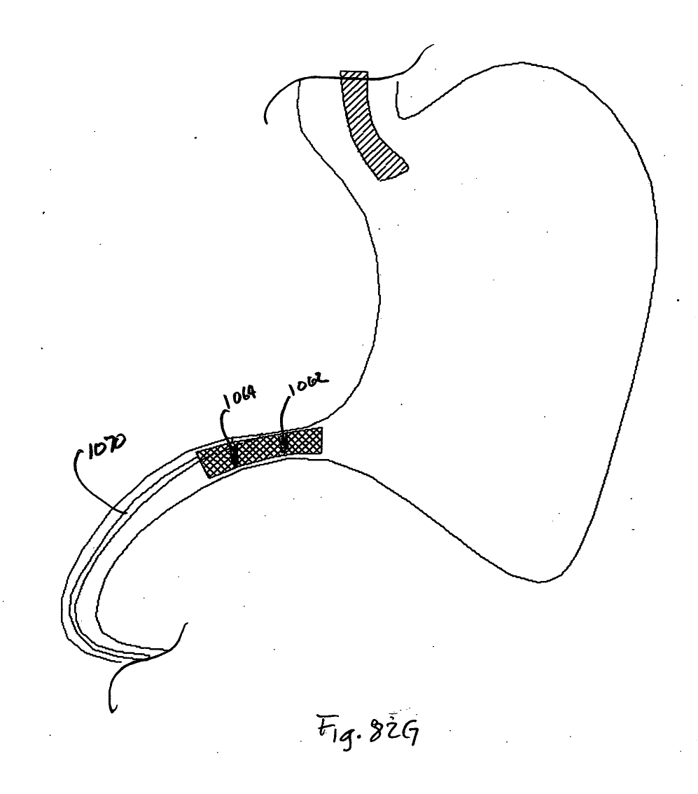

FIGS. 32A and 32B show, respectively, a side, cross-section view and a top, cross-section view of another separator section (551) variation. Adjacent to the duodenum wall (553) is a stent-like structure (555) that is permeable to the stream containing bile and pancreatic fluids. Adjacent to the stent-like structure (555) is a polymeric membrane (557) that defines a volume (559) containing the stent-like structure (555) and is situated to accept the stream containing bile and pancreatic fluids. The volume (559) defined by the polymeric membrane (557) is in fluid communication with the conduit section (561). The interior wall (563) of the polymeric membrane (557) defines a chyme passageway. The polymeric membrane (557) is removable.

FIG. 33 shows a side, cross-section view of another separator section (565) variation. In this variation, a separator wall (567) defines an accumulator volume (569) surrounding the Ampulla of Vater (571). The accumulator volume further contains an absorbent material (575), e.g., typically comprising a spongy foam of the compositions mentioned elsewhere. The accumulator volume (569) is in fluid communication with the conduit member (577).

FIG. 34 shows a side, cross-section view of another separator section (579) variation. In this variation, the region of the duodenal wall (581) surrounding the Ampulla of Vater (583) is depressed with a stent-like cage (585) to form an accumulator volume (587). A separator wall (589) circumscribes the duodenal wall (581) and completes the definition of the accumulator volume (587). The accumulator volume (587) is in fluid communication with the conduit member (591).

FIGS. 35A, 35B, and 35C show, respectively, a side, cross-section view, a top, cross-section view, and a side view of another separator section (560) variation. This variation includes a wall (562) having a shape similar to that of the variation shown in FIG. 26B. In this variation, the accumulator volume (564) formed by the exterior of wall (562) in turn includes a tubing coil (566) having a plurality of openings (568) into which the stream containing bile and pancreatic fluids pass. The tubing coil (566) is in fluid communication with (and, optionally, is an extension of) the conduit section (570). The tubing coil (566) serves to accumulate those fluids and to provide a sink that lengthens the residence time during which the digestive fluids reside in the device before being released in the small intestine.

FIGS. 36A and 36B show respectively, a side, cross-section view and a top, cross-section view of another variation of a separator section (580) and its relationship to a conduit section (582) comprising multiple conduits (584). Each of the multiple conduits (584) opens into the accumulator volume (586).

FIGS. 37A and 37B show, respectively, a side view and a cross section top view of another variation of a separator section (590) and its relationship to a conduit section (592). This variation comprises a blister-shaped wall component (594) that defines a volume that is to be placed about the Ampulla of Vater to collect the stream containing bile and pancreatic fluids for passage to the conduit section (592). The wall component is held in place on the duodenal wall by a number of braces (596) held in place by fasteners (598), e.g., crimped staples, staples, sutures, etc.

FIGS. 38A and 38B show, respectively, a side, cross-section view and a top, cross-section view of another variation of a separator section (600). The separator section (600) comprises a substantially circular wall (602) having an indented blister (604) defining an accumulator volume (606) for surrounding the Ampulla of Vater. The interior surface of wall (602) defines the through-passageway (608) for chyme. The stream containing bile and pancreatic fluids exiting the Ampulla of Vater enters that accumulator volume (606) for passage through the conduit section (610).

The separator section wall (602) comprises a polymeric material with or without strengtheners such as fibers. Suitable polymers are discussed above. The separator section (600) is held in place in the duodenum by an upper ring (612) and a lower ring (614) that are introduced into the chyme passageway (616) of the separator section (600) to press the circular wall (602) against the duodenal wall and affix it there.

FIGS. 39A and 39B show, respectively, a side, cross-section view and a top, cross-section view of another variation of a separator section (620) having a separator wall (622), the interior of which defines a chyme passageway (624) and the exterior of which forms, with the upper inflatable member support (626) and the lower inflatable member support (628) and the duodenal wall, an accumulation volume (632) for the bile and pancreatic fluids exiting the Ampulla of Vater. The upper inflatable member support (626) and the lower inflatable member support (628) may be connected by a bridging member to allow both member supports (626, 628) to be inflated at the same time. The separator wall (622) is fixedly attached to the upper inflatable member support (626) and the lower inflatable member support (628).

FIGS. 40A to 43B show various ways of affixing blister-shaped separator section walls to the duodenal walls.

FIGS. 40A and 40B show, respectively, side, cross-section and top, cross-section views of a separator section (640). The separator section (640) comprises a blister-shaped wall (642) defining an accumulator volume (644) for receiving the stream containing bile and pancreatic fluids exiting the Ampulla of Vater for passage through the conduit section (648).

The separator section wall (642) is maintained in position over the Ampulla of Vater in the duodenum by a pair of struts (650) that extend across the duodenum to a support pad (652) that may be fixed in position by fasteners (not shown) or by pressure against the duodenal wall. The other ends of the struts (650) are connected to support pads (652) that also grasp the edges of the wall (642).

FIGS. 41A and 41B show, respectively, side, cross-section and top, cross-section views of a separator section (660). The separator section (660) comprises a blister-shaped wall (662) defining an accumulator volume (664) for receiving the stream containing bile and pancreatic fluids exiting the Ampulla of Vater for passage through the conduit section (668).

The separator wall (662) is supported by a single bar, stent-like member (670) in turn comprising one or more generally circular members (672) and a number of transverse stabilization members (674). The stent-like member (670) may be made from any of the materials listed above as suitable for stent-like members.

FIGS. 42A and 42B show, respectively, side, cross-section and top, cross-section views of a separator section (680). The separator section (680) comprises a blister-shaped wall (682) defining an accumulator volume (684) for receiving the stream containing bile and pancreatic fluids exiting the Ampulla of Vater for passage through the conduit section (688).

The separator wall (682) is supported in the duodenum by a number of struts (690) passing across the duodenum and each terminated at its remote end by one or more transverse stabilization members (692). At the end of the struts (690) adjacent the separator wall (682) is a support member (694).

FIGS. 43A and 43B show, respectively, side, cross-section and front views of a separator section (700). The separator section (700) comprises a blister-shaped wall (702) defining an accumulator volume (704) for receiving the stream containing bile and pancreatic fluids exiting the Ampulla of Vater for passage through the conduit section (708).

The separator wall (702) is supported in the duodenum by a number of fasteners (710) passing through the duodenum wall. The depicted fasteners (710) are barbed nail fasteners that, after introduction from the duodenum, open and are resistant to removal.

FIG. 44 shows a side, cross-section of another variation of a separator section (710). The separator section (710) comprises a blister-shaped wall (712) defining an accumulator volume (714) for receiving the stream containing bile and pancreatic fluids exiting the Ampulla of Vater.

The accumulator volume (714) further contains a foam material (716) for absorbing those fluids for passage through the conduit section (718).

FIG. 45 is a side view, cross-section of a separator section (720) having bellows (722) in the separator wall (724) allowing the accumulator volume (726) to expand and to contract in response to the expansion and contraction of the duodenal wall during peristalsis and as the bile and pancreatic fluids exiting the Ampulla of Vater pass into that volume (726) and to permit axial flexing during peristalsis. The separator section (720) may be grooved or ridged as desired. The expanded volume passes those fluids through the conduit section (728).

Designs such as that shown in FIGS. 35A-35C and 45 may be used to smooth, to “time-average,” or to delay the flow of bile and pancreatic fluids to the small intestine.

FIG. 46 shows a separator section (750) before and after expansion in a duodenum. Step (a) shows the separator section (750) collapsed and having outwardly facing barb fasteners (752) in vertical furrows or folds (754) in the separator wall (756). The fasteners (752) are for the purpose of affixing the section (750) to the duodenum wall. Step (b) of FIG. 40 shows the expanded section (750) with the barb fasteners (752) extended as they would be when affixed to the duodenal wall. In inflatable balloon may be used to undertake the expansion.

FIGS. 47A and 47B show, respectively, an exploded perspective view of a separator section (770) and a top view of the assembled separator section (770). In this variation, a stent-like structure (772) is placed in the duodenum and expanded to fix it in place on the duodenal wall. An inner continuous membrane member (774) is then introduced on the inner side of the stent-like structure (772) and expanded to affix the continuous membrane member (774) to that stent-like structure (772). In this variation of the separator section (770), the components providing upper and lower sealing functions are not shown. They may be situated in the upper section or the lower sealing section, as discussed above.

FIG. 47B shows the placement of the inner continuous membrane member (774) in the stent-like structure (772).

Because the stent-like structure (772) provides some volume for accepting the bile and pancreatic fluids, a separate volume for those fluids may not be desired. FIG. 41A, however, shows an optional separate collection volume (776) for accepting those fluids. The volume (776) may then be attached to the conduit section (778).

FIG. 48 shows a separator section (773) comprising an ion-permeable membrane (775) separating the chyme passageway (777) from the surface (779) adjacent the bile and pancreatic enzymes. The ion-permeable membrane (775) may be selected to allow water to pass, e.g., a polyimide membrane, or water and bicarbonate to pass, e.g., a regenerated cellulose membrane. Larger molecules, such as those comprising the bile and pancreatic enzyme stream do not pass through the membranes. The choice of a suitable membrane for such service is readily made using prior art information.

FIG. 49 shows a schematic top-view cross-section of a separator section (781) having a circumferential, semipermeable membrane (783) selected to allow water and (optionally) bicarbonate ions to pass from chyme into the annular space (785) and then to the small intestine. Additionally, a small chamber (787) is formed of an impermeable membrane (789) surrounding the Ampulla of Vater and is fluidly connected to the conduit section.

FIG. 50 shows a separation section (1074) having several radio-opaque markers useful in properly placing the section (i8) during implantation. The separation section (i8) includes an aperture (1090) for isolating the Ampulla of Vater bracketed by a proximal marker (1092) and a distal marker (1094) and a axial marker (1096) aligned with the center of the aperture (1090).

### Lower Sealing Section

As noted above, my device employs a lower seal to prevent mixing of chyme with bile and pancreatic fluids until those digestive fluids exit the conduit section.

FIGS. 51-63 show various seal configurations for use in the lower seal section.

FIG. 51 shows a rubbery tubular seal (800) residing in a seal groove (802) in the wall (804) of the lower seal section. The wall (804) provides pressure against the tubular seal. The passageway (810) in the rubbery tubular seal (800) may be filled with a gas, a pressurized gas, or a liquid. Depending upon the material chosen for the rubbery tubular seal (800), the passageway (810) may be open to the local environment. The seal (800), typically comprising an elastomer, then must remain expanded for sealing purposes.

FIG. 52 shows a coiled spring seal (814), optionally covered or coated with a membrane, also residing in a seal groove (802) in the lower seal section wall (806). The spring found in seal (814) may comprise a material selected from biocompatible polymers, metals, alloys, or their mixtures selected to maintain the seal (814) in an open condition and to prevent chyme and digestive fluids from passing.

FIG. 53 shows a multi-layer bellows seal (818) also residing in a seal groove (802) in the lower seal section wall (806).

FIG. 54 shows a multi-seal assembly having two fixed seal members (830, 832) in which the inter-seal area (834) is drained by passageway (836) that flows into the conduit section (838). This secondary drain improves the overall efficiency of the lower seal section.

FIG. 55 shows a multi-seal assembly having a first fixed seal member (840) and a second fixed, foam, compliant seal member (842). The width of the second seal (842) and its relative softness provides a high sealing efficiency. The first seal (840) may be narrow, typically is less resilient and is effective in preventing the leakage or flow of slurries such as chyme.

FIG. 56 shows a multi-level seal (848) having a number of seal wiper levels (850). The opening to the conduit section (850) may also be seen.

FIGS. 57A and 57B show, respectively, a perspective, cross-section view and a side, cross-section view of a multi-wiper seal (854) having drainage between the seal wipers (856). The drainage openings (858) pass into a plenum (860) and join with the major passageway (862) for the bile and pancreatic fluids. The passageways together communicate with the conduit section (864).

FIG. 58 shows a seal (870) comprising a membrane (872) that is crimped and pulled into a channel (876) in the lower seal section wall (878) by a tightening loop (874).

FIG. 59 shows a seal (880) having a corrugated facing (882) and a hydrogel or other soft polymeric covering (884) on a base seal component (886). The seal assembly (880) typically is circumferential or continuous to exert a radial force upon the duodenal wall.

FIG. 60 shows a seal assembly (890) having a distensible outer layer (892) and an inner chamber (894) containing a fluid. The outer diameter of the distensible outer layer (892) may be adjusted by changing the amount of fluid contained within inner chamber (894). Typically the shape of the seal assembly (890) is a very thin donut.

FIG. 61 shows another seal assembly (898) having an outer seal component (900) and a seal spring (904) sized to maintain outward pressure on the outer seal component (900), maintain its shape, and maintain pressure on the duodenal wall. The outer seal component (900) has a rounded cross-section that provides a small contact patch with the duodenal wall but allows ease of movement on that wall if the design requires such movement. The seal assembly (898) is generally circumferential and resides in a seal channel or groove (906). The seal spring (904) need not be continuous.

FIG. 62 shows another seal assembly (910) having an outer seal member (912) and a stent-like wire spring (914) providing pressure against the outer seal member (912). In this variation, the seal member (912) is generally rectangular in cross-section and has a broad contact patch with the duodenal wall. With a broad contact patch, the pressure of the outer seal member (912) against the duodenal wall may be lessened without diminishing the sealing capabilities of effectiveness of the seal assembly (910).

FIG. 63 shows an inflatable seal assembly (920) that utilizes chyme to inflate a seal member (922) only during the period that chyme is being released from the stomach. The seal member (922) may be a flaccid tubing with interior inflation volumes (924) and having one or more chyme passageways (924) opening to, and in fluid communication with, the interior (928) of the device. The seal member (922) may be constructed with a bias so that it collapses or flattens and expels chyme from the interior inflation volumes (924) in doing so. The FIG. 54 shows a diverter sheet (928) that creates an open volume (930) that collects an amount of chyme to assist in inflating the seal member (922) when chyme is present in the device.

FIG. 64 shows a side view, cross section view of a lower seal section (919) in which the active seal (921) comprises an extended polymeric foam material. The foam material is biocompatible and may be either open or closed cell, although the sealing effectiveness is more pronounced with closed cell foam allowing physically smaller seals. The seal (921) may be glued to the separator wall membrane (923).

FIG. 65 shows another version of a lower seal section (925) having a plurality of “O” rings (927) embedded in a polymeric matrix (929). The “O” rings (927) are selected to provided pressure against the duodenal wall (931). The polymeric matrix (929) is a physical continuation of the separator wall membrane (933).

FIG. 66 shows another version of a lower seal section (935) comprising a circular coiled spring (937) similar to those shown in FIGS. 62 and 63. In this variation, the spring (937) is embedded in a polymeric matrix (939) but is aligned so that it provides a constant pressure against the duodenal wall (941). Again, the polymeric matrix (939) may be a continuation of the separator wall membrane (943) although it need not be.

### Conduit Section

The conduit section (210 in FIG. 4), as noted above, comprises one or more conduit tubing members in fluid communication with the collection volume associated with the separator section and has as its primary function the step of transporting the separated bile and pancreatic fluids to or towards the Ileum for release there. If multiple conduit tubing members, they may be of the same or differing lengths. Each conduit member may be formed of a single biocompatible material variously biodegradable or non-biodegradable or may be formed of two or more different biocompatible materials that may each be biodegradable or non-biodegradable in various physical configurations. Depending upon the course of treatment desired, the conduit members may be designed to have a finite life in the digestive track before dissolution or may be designed for continuous life until physically extracted. As noted below, the conduit members may be designed for partial dissolution, e.g., to shorten the length(s) of the conduit member(s) at a selected interval during a treatment regimen. The conduit members may include porous sections, sections comprising ion-permeable membranes, sections having openings of significant size, or the like depending upon the treatment envisioned.

The conduit members may include components to prevent sludge or salt formation or components to remove those obstructions if they should occur.

The conduit members may include other ancillary features to provide radio-opacity, to prevent kinking, buckling, or other form of obstruction, or to alleviate the effects of any such obstruction.

The conduit section may comprise a single member (e.g., 470 in FIGS. 26A and 26B) or may comprise multiple members (e.g., 584 in FIGS. 36A and 36B). The conduit section may perform only the simple function of passing bile and pancreatic fluids from the collection volumes to the terminus (or termini) of the conduit member or members or may perform other ancillary functions, e.g., fluid storage or programmed dispersal of the fluids. If desired, the conduit members may be removable for a variety of reasons, e.g., simple replacement of the component or for revising a course of treatment by replacement of one or more conduit members with members of a different length thereby changing the course of patient treatment. FIGS. 55-58 show examples of removable and replaceable conduit members and their manners of attachment.

FIG. 67 shows a quick disconnect snap conduit connection assembly (900) in which the male portion (902) fits into the female section (904) and is secured in position by a number of detent balls (906) fitting within the annular groove (908). The detent balls (906) may be spherical, reside in openings within the male portion (902), and made to protrude from that male portion (902) by springs or the like. The detent balls (906) may be hemispherical and formed in place exterior to the male portion (902) wall. In the latter instance, the male portion (902) and the female portion (904) should plastically deform to allow engagement and disengagement upon axial movement.

Either of the male portion (902) or the female portion (904) may be chosen to constitute the removable portion and, conversely, the stationary portion or base.

FIG. 68 shows another quick disconnect snap conduit connection assembly (910) in which the male portion (912) fits into the female portion (914) and is secured in position by an exterior circumferential collar (916) fitting within the interior annular groove (918) found in the female portion (914). The collar (916) may be formed in place exterior to the male portion (912) wall. Again, at least one of the male portion (912) and the female portion (914) should plastically deform to allow engagement and disengagement upon axial movement between the two portions.

FIG. 69 shows a connection assembly (920) having a magnetic base portion (922) with a mating surface (924) with multiple connector barbs (926) for connecting the base portion (922) to the duodenal wall. The opening (928) in the center of the base section (922) is for sitting the base section over the Ampulla of Vater. The mating surface (924) is shown to be substantially flat but, of course, may have any convenient surface shape that matches the mating surface (934) of the removable portion (930).

The removable portion (930) includes that mating surface (934) which may be magnetized or may be simply attracted to a magnet, e.g., the mating surface (934) may be formed upon a structure comprising a ferromagnetic metal. The removable portion (930) may also comprise a passageway (932) for passage of the bile and pancreatic fluids down the length of the conduit (938) into the small intestine. A mating lip (936) is shown surrounding the removable portion (930) mating surface (934) that fits around the stationary base portion (922) mating surface (934) and stabilize the relative positions of the two portions (922, 930) after implantation and during use.

FIG. 70 shows another variation of a magnetic connection assembly (940) having a base portion (942) and a removable portion (944) connected to the conduit member (946). The base portion (940) is shown to have a blister shape enclosing a volume (948) that may be situated around and over the Ampulla of Vater. The magnet (or magnetizable metal or alloy) component (950) may be placed surrounding an opening (952) that matches a similar opening (954) having a magnet (or magnetizable metal or alloy) component (956) in the removable portion (944). Clearly, in this variation, one or more of the magnet (or magnetizable metal or alloy) components (950, 956) must be a magnet and the other must be a magnet or magnetizable metal or alloy for the connector assembly (940) to remain connected.

FIGS. 71A and 71B show a variation of one portion of a magnetic coupling assembly, a base portion (945) or retainer that may be affixed to the duodenal wall and connected to the proximal end of a conduit (944) such as is seen in FIG. 58. This variation includes a ring section (947) with a number of legs (949) that are operative to pierce the duodenal wall and split and form an anchor. The opening (951) in the ring section (947) may be centered over the Ampulla of Vater to collect bile and pancreatic fluids. The ring section (947) must comprise a magnet or magnetizable metal or alloy for a cooperating connector assembly to remain connected.

FIGS. 72 to 79 show variations of the conduit that may be fixed to the other portions of the device or duodenal wall as otherwise discussed here or may be detachable.

FIG. 72 shows a conduit comprising a simple tubing member (950). The tubing member (950) may have a constant diameter and wall thickness from one end of the conduit to the other or may have varying or stepped dimensions as desired. The cross-sectional shape of the tubing member may be circular, oval, square, hexagonal, or other desired shape. The composition of the tubing member may comprise any convenient material, usually one or more biocompatible polymers, often selected from the polymer lists provided above. Depending upon the course of treatment selected, the tubing member may be partially or completely biodegradable or non-biodegradable.

FIG. 73 shows a composite tubing member (952) comprising sections having different compositions, e.g., a non-biodegradable polymer (954) and a biodegradable polymer (956). The designer for a specific device utilizing the principles and disclosure of this application may use multiple compositions for a variety of specific purposes. One such purpose would be to select a biodegradable polymer composition having specific physical sizes allowing a medical practitioner to select a conduit having a specific residence time in the digestive tract. That is to say that the tubing member would dissolve after a chosen time and no longer transport bile and pancreatic fluids distally into the small intestine thereby terminating the treatment. The medical practitioner could choose a conduit having a section of biodegradable polymer situated in the mid-course of the conduit, the biodegradable section selected so that upon its dissolution, the overall conduit length becomes shorter, thereby lessening the intensity of the treatment.

FIG. 74A-74C shows a length of conduit (960) having a closable access port (962) allowing access to the interior of the conduit (960) in the event that cleaning or clearing of blockage is needed. The access port (962) is shown to have a movable closure flap (964) that, in this variation, is simply secured to the conduit wall on a side of the flap (964) by an adhesive (966) or the like. This arrangement allows a medical practitioner to utilize a catheter/guidewire combination to access the interior of the conduit (960) by pressing against the exterior of the flap (964). Upon removal of the catheter/guidewire, the flap (964) should self-close and prevent the entry of chyme into the interior of the conduit.

FIGS. 75A and 75B show a length of conduit (970) including a polymeric wall (972) and one or more stiffeners (974). The stiffeners (974) may comprise an independent material or component, e.g., a wire or cable, operable to maintain the conduit in substantially the same position as implanted. The stiffeners (974) may alternatively comprise the same or similar material relying upon the difference in cross-section to provide axial stiffness or the difference in inherent stiffness between the stiffener (974) and the conduit wall to provide axial stability. For instance, if the tubing forming the conduit (970) is extruded of a single material with a cross-section such as shown in FIG. 75B, the shape of the so-extruded stiffener (974) will provide length-wise shape stability. If the stiffeners (974) are co-extruded of a material having a comparatively higher stiffness, the stiffening effect is enhanced.

The number of stiffeners (974) placed in the conduit (970) may be one or more and are or the purpose of providing shape stability, whether that shape is linear or curved.

FIG. 76 shows a length of conduit (980) that includes a stripe (982) of radio-opaque material in the conduit wall (984). The stripe (982) allows visualization via x-ray of the positioning of the conduit (980) in the digestive tract without hiding the contents of the conduit (980). The radio-opaque material, for instance, may be mixed with and coextruded with the conduit tubing. Suitable radio-opaque materials include fine particulates of barium sulfate, bismuth oxychloride, bismuth subcarbonate, bismuth trioxide, tungsten, gold, tantalum, and Platinum Series metals such as platinum.

FIG. 77 shows a conduit terminus (986) having a widened region, specifically, a bell shape (988). Such a shape lessens the chance that a blockage will form in that region of the conduit.

FIG. 78 shows a conduit section (990) that is coiled. Such a configuration may be used to allow the conduit section (990) to unfurl or uncoil as it fills with bile and pancreatic fluids and, to some extent, to self-deploy. If not used in that way, the coil may be used to provide time-delay storage for bile and pancreatic fluids.

FIG. 79 shows a section of conduit (1002) having a number of slits (1004) communicating between the interior passageway (1006) and the outer surface (1008). These slits (1004) may be employed in a design to provide a relief in the event that the conduit becomes blocked.

FIG. 80A shows a section of conduit (1010) having a conduit wall (1012) with a number of duckbill-style valves (1014) that may be used for various design purposes, e.g., to allow passage of a selected amount of the bile and pancreatic fluids in internal passageway (1016) to the exterior surface (1018) as an object of the obesity treatment or to allow passage of that fluid mixture out of the conduit (1010) in the event that the internal passageway (1016) becomes partially or completely obstructed downstream of the duckbill-style valves (1014).

FIG. 80B shows a partial, side-view, cross-sectional view of the duckbill-type valve (1014) with one of its “bills” (1020) and the external opening (1022) of the valve (1014).

FIG. 80C shows a partial, side-view, cross-sectional view perpendicular to the view shown in FIG. 80B. In particular, this view of the duckbill-type valve (1014) shows both “bills” (1020) of the valve, the external opening (1022) of the valve (1014), and its positioning in the conduit wall (1012). This type of valve allows fluid found in the conduit passageway (1016) to exit the conduit section (1010) when a design pressure differential between the interior and the exterior of the conduit section (1010) is attained. The valve does not permit the reverse flow of fluids from the exterior to the interior passageway (1016) of the conduit section (1010).

### Methods of Deployment

Described below are a number of installation devices and methods suitable for deploying the devices discussed above. My devices may be introduced intraorally, endoscopically without the need for any open surgery.

The general sequence of implantation includes the following steps generally in the following sequence. First, the distal tip of the conduit or conduits is advanced to the desired site in the Ileum. The separator section and, often, the lower seal section is then fixed or positioned for subsequent fixation in the duodenum. The separator section is positioned to maintain separation of the chyme from the digestive fluids issuing from the Ampulla of Vater. Typically, the implantation of the device is concluded by affixing the upper section to the stomach or pylorus.

FIGS. 81A, 81B, 81C, and 81D show a first variation of an installation system for my device. This system employs a guide member (1030), in structure and function similar to a guidewire, to deploy the conduit's (1032) distal tip to the jejunum or Ileum. The guide member (1032) includes an interior passageway (1034) for passing an inflation fluid to an expandable member or balloon (1034) located at the distal tip of the guide member (1030). The guide member (1030) will typically be about 2-3 meters in length. The expandable member (1036) is typically compliant. Compliant expandable members expand and stretch with increasing pressure and may comprise polymeric materials such as one or more of the Silicones, thermoplastic elastomers (TPEs), and polyethylene or polyolefin copolymers. The expandable member (1036) may be noncompliant if the designer so chooses. Non-compliant expandable members may comprise suitable polymeric materials such as polyethylene terephthalate (PET) or polyamides, and remain substantially at a pre-selected diameter as the internal pressure increases beyond that required to fully inflate that expandable member (1036).

Compliant polymeric materials provide a degree of softness to the member that aids its passage through, and expansion within the digestive tract. Such compliant polymeric materials often display good abrasion and puncture resistance at the thicknesses typically used in medical devices.

The guide member (1030) includes a passageway (1034) through which inflation fluid is passed to the inflation member (1036) through openings (1038) in the wall of the guide member (1030). The passageway (1034) is proximally attached to an inflation and deflation mechanism, e.g., a compressor or compressed gas source or a liquid pump for inflation of the inflation member (1036) and, optionally, a vacuum source for deflation of the inflation member (1036).

The inflation member (1036) serves several functions. Partially inflated, the inflation member (1036) serves as a dead weight during insertion of the inflation member (1036) into the duodenum, jejunum, and all the way through to the Ileum. This dead weight allows ease of maneuvering through the tortuous small intestine, particularly under fluid pressure in the intestine. The inflation member (1036) may alternatively be filled with normal saline or a radiographic contrast fluid. Use of such contrast fluid aids in locating the distal tip of the guide member (1030) under fluoroscopy

After the distal tip of the guide member (1030) is maneuvered to an appropriate location in the jejunum or Ileum, the inflation member (1036) is further inflated to anchor the distal tip of the guide member (1030) at that location as the conduit member (1032) in FIG. 81A or (1042) in FIG. 81B) and the separator section are deployed. By inflating the inflation member (1036) further, the expanded inflation member (1036) tightly fits within the lumen of the jejunum or Ileum and acts to anchor or to secure the distal tip at that location.

The guide member (1030) may include radio-opaque markings, e.g., bands (1040) at the proximal end of inflation member (1040), to help visualize the location of the inflation member (1030) during placement. Such radio-opaque markings may be placed at any site on guide member (1030) the designer considers appropriate for this function.

FIG. 81A shows the guide member (1030) passing through a simple conduit member (1032), i.e., a conduit member (1032) having a single central passageway for digestive fluids. FIG. 81B shows the guide member (1030) passing through a conduit member (1042) that includes a separate guide member passageway (h8). The guide member passageway (0144) includes an opening (1046) into the chyme passage of the separator section (1048) in FIG. 81B) allowing easy access from the chyme passageway and ease of guide member (1030) removal. The guide member passageway (1044) is isolated from the digestive fluids passageway (1050) as readily seen in FIG. 81C.

### Method of Deployment

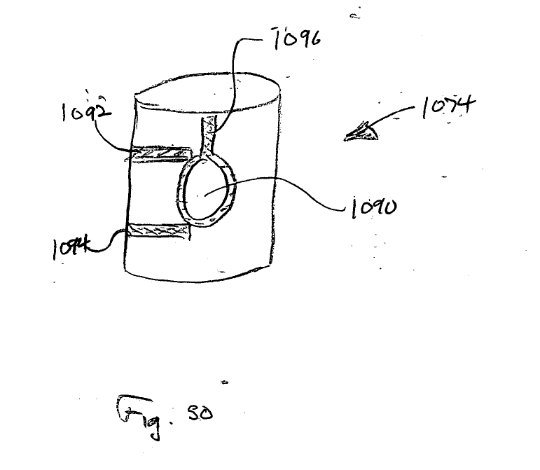

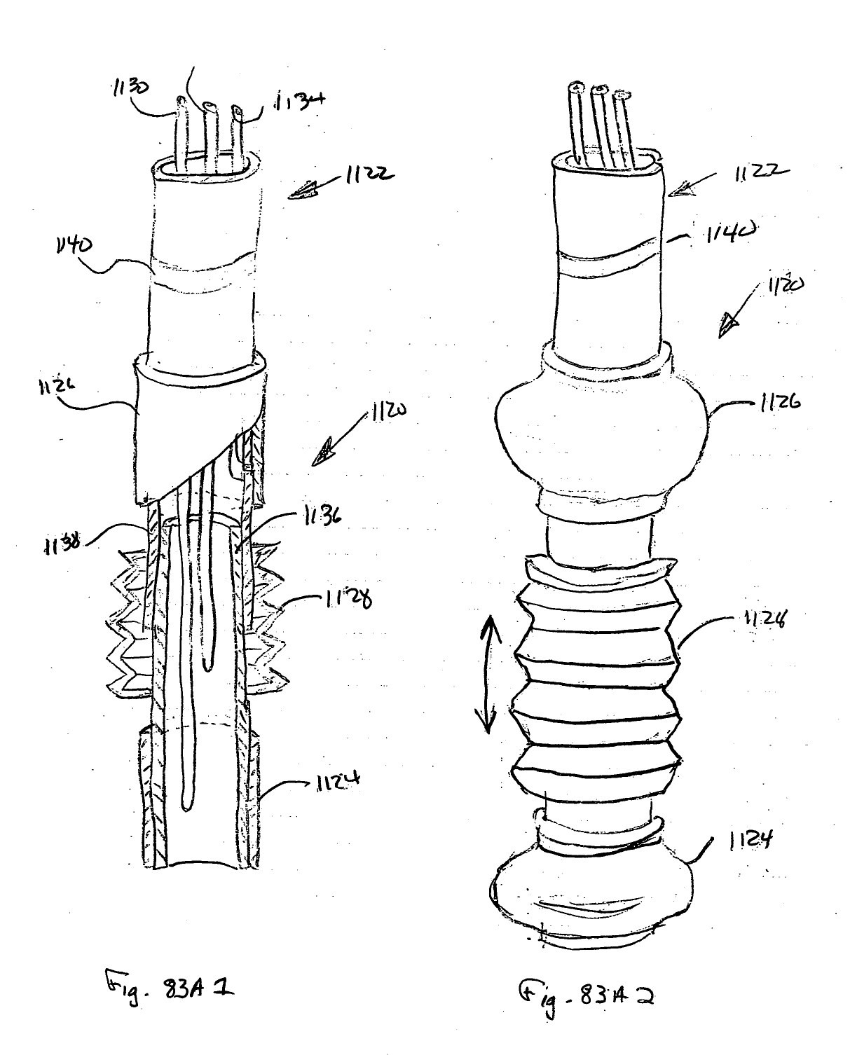

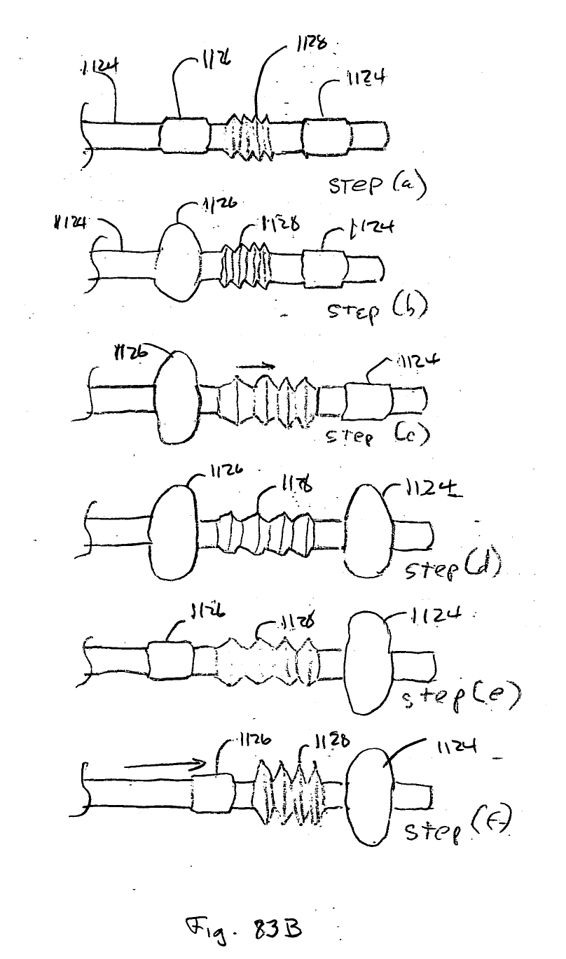

An implantation method using an endoscope and the guide member shown in FIGS. 81A-81D is schematically shown in FIGS. 82A-82G.

As shown in FIG. 82A, an endoscope (1060) is passed down to the level of duodenum (i2) through the mouth. As shown in FIG. 82B, a guide member (1066) of the type shown in FIGS. 81A-81D is then passed through the channel (1064) of the endoscope (1060) and advanced into the duodenum (1062) and through the length of the jejunum. In FIG. 82C, the expansion member (1068) of the guide member (1066) is filled with saline or a radiographic contrast fluid to act as a dead weight to maneuver the guide member (1066) through the tortuous jejunum and Ileum. The radiographic contrast material also helps the user to visually follow the progress of the guide member (1066) under fluoroscopy. After confirming the location of the distal end of the guide member (1066) in the distal jejunum or Ileum by the use of either fluoroscopy or by direct visualization from the endoscope, as shown in FIG. 82D, a conduit member (1076) is advanced by threading the conduit member (1076) over the guide member (1066).

After the distal end (1072) of the conduit member (1066) reaches the level of the anchored inflation member (1068), the deployment of the separation section (1074) is begun.

FIG. 82E shows the extension of a balloon catheter (1078) from the endoscope (1060). FIG. 82F shows placement of the separator section (1074) in the duodenum (1062).

The predeployed configuration of the separation section (1074) shown in this procedure includes three distinct visual markers that can be seen through the endoscope. This variation of the separation section (1074) used in this example of the procedure may be seen in FIG. 50. These markers help guide the separation section (1074) to the right location so that the aperture (1090) of the separation section (1074) is positioned at the level of and adjacent to the Ampulla of Vater. The proximal marker (1092) and the distal marker (1094) allow the axial positioning of the separation section (1074) at the Ampulla of Vater. One of these markers is the distal marker (1094) and the other is a proximal marker (1092). While positioning the separation section (1074), the distal marker (1094) is to be guided distal to the Ampulla of Vater and the proximal marker to lie proximal to the Ampulla of Vater. The third marker (1096) is a “laterality marker” that ensures that the aperture (j1) opens onto the Ampulla of Vater. After aligning the side of the aperture (1092) with the Ampulla of Vater, the separation section (1074) is deployed by either inflating the inflation member (1068) or by activating another deployment mechanism. The inflation member (1068) is then deflated, leaving the device in vivo. As shown in FIG. 82G, after deployment, the inflation member (1068) at the tip of the guide member (1066) is deflated and removed. The endoscope (1060) may then advanced into the separation section (1074) to visually confirm that the aperture (1090) is properly located surrounding the Ampulla of Vater.

FIGS. 83A1, 83A2, and 83B shows another implantation variation. In this instance, the device (1120) is included as an integral, distally located portion of a conduit member (1122). The device includes three expandable members or balloons, a distal radially expandable member (1124), a proximal radially expandable member (1126), and an axially expandable motive member (1128). FIG. 83A1 is a partially cutaway side view of the variation with each of the expandable members ((1124), (1126), and (1128)) in a deflated condition. FIG. 83A2 is a side view of the variation (1120) with each of the expandable members ((1124), (1126), and (1128)) in an inflated condition. Each of the expandable members ((1124), (1126), and (1128)) is independently supplied by an inflation/deflation conduit ((1130), (1132), and (1134)). As the axially expandable motive member (1128) expands and contracts along the axis of the variation, an inner slider portion (1136) slides back and forth within an outer support member (1138). The section (1120) may also include one or more radio-opaque markers (1140).

FIG. 83B shows the procedure for using the device to “walk” the motive variation (1120) through the small intestine. In step (a), the distal end of the conduit member (1122) is inserted in the lumen of the small intestine. In step (b), the proximal expansion member (1126) is inflated to temporarily anchor the shaft (1142) in place. In step (c), the axially expandable motive member (1128) is inflated to expand the member (1128) forward. In step (d), the distal expandable member (1124) is inflated to fix the distal end of the device in place.

In step (e), the proximal expandable member (1126) is deflated allowing the axially expandable motive member (1128) to contract. This contraction may take place due to spring members restoring the axially expandable motive member (1128) or to a suction applied through inflation/deflation conduit (1132). The contraction of the axially expandable motive member (1128) carries the more proximal portions of the conduit member (1122) along with it.

This procedure is repeated as often as is necessary to place the distal end of the conduit (1122) in the proper region of the Ileum for the treatment mentioned above.

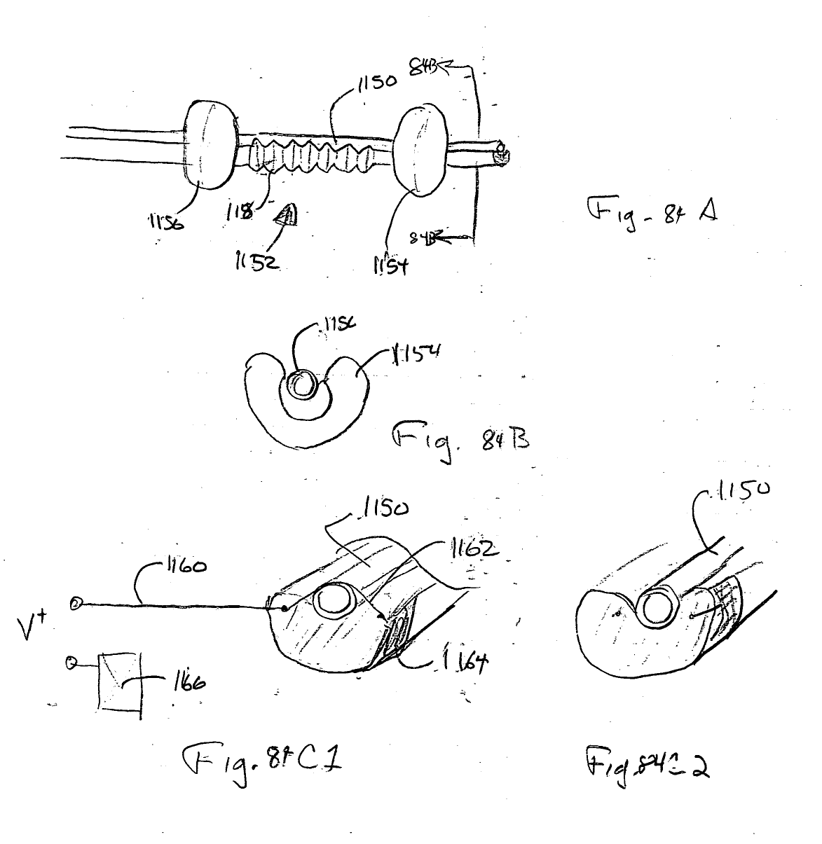

FIGS. 84A, 84B, 84C1, and 84C2 show a device that is similar in the principles of operation to the device shown in FIGS. 83A1, 83A2, and 83B. The structure is not integral with the conduit, however, and simply carries the distal end of the conduit member (1150) along the intestine until the conduit member is released from the carrier (1152).

FIG. 84A is a side view of the carrier device (1152) showing the distal expandable member (1154), the proximal expandable member (1156), and the axially expandable motive member (1158). The conduit member (1160) may also be seen.

FIG. 84B shows an end view of the device (1152) and the distal expandable member (l3) cradling the conduit member (1150). The shape of the distal expandable member (l3) is shown to be approximately ¾ of a donut when expanded. This allows substantial, but centered contact of the expandable member (1154) with the intestine wall as it moves along that wall transporting the conduit member (1150).

FIGS. 84C1 and 84C2 show a simple but effective manner of electrolytically releasing the conduit member (1150) from the carrier (1152) so that that carrier (1152) may be removed. The electrical conductors include at least a more “noble” metal wire (1160), e.g., platinum, and a less “noble” metal wire (1162), e.g., tungsten. The less noble metal wire (1162) may be quite thin, e.g., 0.002″ to 0.015,″ for quick detachment upon application of a modest voltage; the less noble metal wire (1162) is used to hold the conduit member (1160) in place on the carrier until release. The version shown in FIGS. 84C1 and 84C2 includes a plate (1168) to increase the surface area for flow of electrical current through the conductive fluids in the intestine. The circuit includes a skin patch (1166) to complete the circuit.

A modest voltage, e.g., 6-24 volts DC, is applied to the terminals, flows through the more noble conductor (1160), and through less noble conductor (1162) where it electrolytically erodes until it eventually breaks and releases the conduit member (1150). The erodible less noble conductor (1162) does not appreciably heat during imposition of the voltage. Until that break occurs, the voltage continues to flow though the plate (1164), through the liquid intestinal contents, to the skin patch (1166), and back to the current source.

The carrier (1152) may then be retracted and removed from the patient leaving the conduit member (1150) in place.

In other variations of the implant procedure, the distal tip of the conduit member may be advanced to the selected site in the distal jejunum or Ileum by a releasable or severable attachment to the advancing tip of an endoscope.

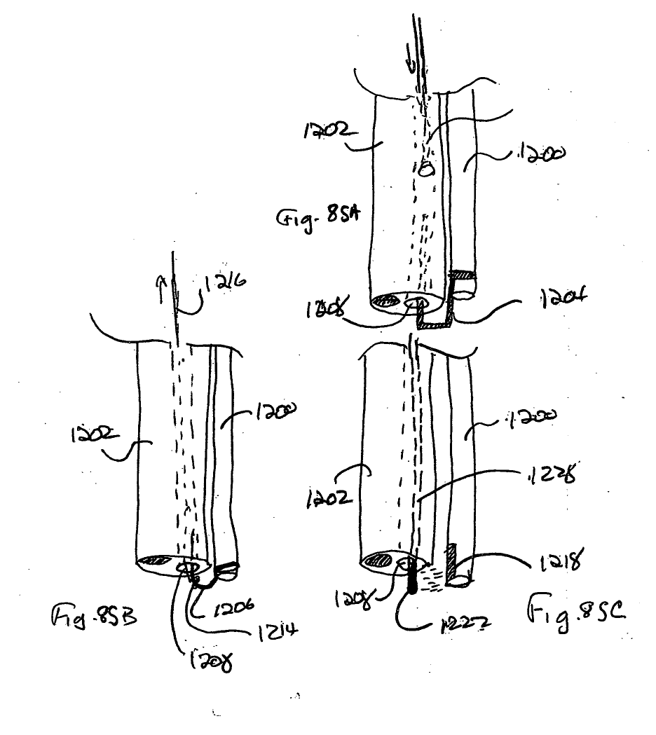

FIGS. 85A-85C show a number of attaching elements operable to temporarily connect the distal end of a conduit member (1200) to the distal end of an endoscope (1202) during the transit to the small intestine. FIG. 85A shows a mechanical hook (1204) that enters the endoscope channel (1206). An optional mechanical pusher (1212) extending back to the proximal end of the endoscope channel (1208) may be used to dislodge the mechanical hook (m3) from the endoscope. FIG. 85B shows another mechanical hook (1206) that is held in position by a slip-knot (1214) in a filament (1216) extending back to the proximal end of the endoscope. Pulling on the filament (1216) unties the slip knot (1214) allowing the mechanical hook to leave to endoscope channel (1208). FIG. 85C shows a magnetic connection formed of a magnet (or a plate comprising a ferromagnetic material) (m1218) situated on or near the distal end of the conduit member (1200) and a magnet (1222) situated on the distal end of a tool (1226) that passes through the endoscope channel (1208). Withdrawing the magnetic tool (1226) through the endoscope channel (1208) breaks the magnetic connection between the tool (1226) and the magnetic site (1218) on the conduit member (1200) and releases that conduit member (1200). The endoscope may then be withdrawn from the patient.

I claim as my invention:

1. A device operable to provide substantial isolation of chyme from bile and pancreatic enzymes for a portion of a human digestive system, that portion extending from the pylorus of that digestive system to one or more selected sites in the small intestine, the device comprising:
an upper portion attachable to a distal portion of the stomach operable to at least partially support the device in the digestive system,
a separator section having a separator wall and having at least one passageway with proximal and distal ends and passageway walls, the at least one passageway operable to accept chyme at the proximal end, to discharge chyme at the distal end, and the passageway walls operable to collect bile and pancreatic enzymes for delivery to at least one conduit for transport to the at least one selected site in the small intestine, to maintain those bile and pancreatic enzymes in substantial isolation from chyme, and to allow contact of that chyme with small intestine walls, and
the at least one conduit operable to transport substantially all of the collected bile and pancreatic enzymes to the at least one selected site in the small intestine.

2. The device of claim 1 wherein the separator section further comprises at least one distally located seal operable to maintain separation of the chyme from the bile and pancreatic enzymes at that distal end.

3. The device of claim 1 wherein the separator section further comprises at least one proximately located seal operable to maintain separation of the chyme from the bile and pancreatic enzymes at that proximal end.

4. The device of claim 1 where the upper section is removably attachable to the pylorus.

5. The device of claim 1 where the upper section is removably attachable to a distal portion of the stomach.

6. The device of claim 1 wherein the upper section further comprises at least one distally located seal operable to maintain separation of the chyme from the bile and pancreatic enzymes at that distal end.

7. The device of claim 1 where the upper section further comprises at least one proximally located seal operable to maintain separation of the chyme from the bile and pancreatic enzymes at that proximal end.

8. The device of claim 1 wherein the conduit section comprises a fixed base and a cooperating removable fixture for removable attachment to the fixed base.
